(19) United States
(12) Patent Application Publication
(10) Pub. No.: US 2024/0181091 A1
(43) Pub. Date: Jun. 6, 2024

(54) ORAL DELIVERY OF SILICA/ALUMINOSILICATE NANOPARTICLES

(71) Applicant: Cornell University, Ithaca, NY (US)

(72) Inventors: Thomas C. Gardinier, II, Raritan, NJ (US); Jacob A. Erstling, Ithaca, NY (US); Nirmalya Bag, Ithaca, NY (US); Ulrich B. Wiesner, Ithaca, NY (US)

(21) Appl. No.: 18/551,786

(22) PCT Filed: Mar. 21, 2022
(86) PCT No.: PCT/US22/21220

§ 371 Date: Sep. 21, 2023

Related U.S. Application Data
(60) Provisional application No. 63/163,898, filed on Mar. 21, 2021.

(51) Int. Cl. A61K 49/00; A61K 47/69

(52) U.S. Cl. CPC A61K 49/0065; A61K 47/6923; A61K 47/6929; A61K 49/0017

(57) ABSTRACT

Compositions for oral delivery of silica nanoparticles and/or aluminosilicate nanoparticles, and methods of use thereof. The individual nanoparticles comprise a silica core or an aluminosilicate core and, optionally, polyethylene glycol (PEG) groups each disposed on a surface of the nanoparticle. In various examples, the nanoparticles have a size of about 20 nm or less. In various examples, the nanoparticles can be delivered to an individual's gastrointestinal tract, pass through the mucus layer thereof, and permeate the epithelial lining thereof. In various examples, the nanoparticles comprise various targeting, diagnostic, or therapeutic group(s) and can retain one or more said activit(ies) after delivery. Methods of oral delivery and methods of treating a disease can comprise orally administering composition(s) to an individual. Non-limiting examples of diseases include infections, cancers, neurological conditions/discases, neurodegenerative discases, psychological conditions/diseases, inflammatory conditions/diseases, cardio-vascular diseases, and the like.

### CROSS-REFERENCE TO RELATED APPLICATIONS

This application claims the benefit of U.S. Provisional Patent Application No. 63/163,898, filed Mar. 21, 2021, the contents of the above-identified application are hereby fully incorporated herein by reference in their entirety.

### STATEMENT REGARDING FEDERALLY SPONSORED RESEARCH

This invention was made with government support under grant number CA199081-01 awarded by the National Institutes of Health. The government has certain rights in the invention.

### BACKGROUND OF THE DISCLOSURE

The route of administration of therapeutic drugs to patients plays a significant role in patient disease outcome. In particular, oral administration can be advantageous compared to intravenous administration as it is typically associated with higher patient compliance and with an enhanced capability for repeat dosing. The last two decades have seen substantial efforts in nanoparticle-based drug delivery. In contrast to small molecule therapeutics, however, in the case of nanoparticles the oral delivery route has been significantly hindered by the fact that most nanoparticle platforms are unable to be effectively absorbed through the gastrointestinal (GI) tract into the bloodstream. Two major barriers that any orally delivered therapeutic must be able to overcome are the dense mucosal layer covering the epithelial lining as well as the epithelial lining itself.

The mucus layer composed of a heterogenous mixture of diverse protein and saccharide species functions as a key barrier in the human body against disease and foreign objects such as nanoparticles. The mucus constituents participate in a myriad of interactions with these foreign objects to prevent their entry and passage. These include electrostatic interactions, size-based exclusion, surface aggregation of proteins, and van der Waals interactions. All these interactions, often collectively, hinder diffusion and hence permeability of the foreign materials across the mucosal layer. Recently, a large body of biophysical studies evaluated nanoparticle diffusion in mucus as a first step to establish nanoparticles of suitable size that can relatively rapidly diffuse through mucus barriers. However, most of the work focused on nanoparticles in the >100 nm size range.

The epithelial lining acts as a second significant barrier and is mostly made up of enterocytes that form tight junctions making it a strong obstacle for the oral administration of nanoparticles. A commonly utilized assay to study the permeability of drugs and nanoparticles across the epithelial lining is the Transwell permeability assay. This industry-standard assay uses a human colorectal cancer Caco-2 cell line that forms a monolayer of cells with a structure similar to that of enterocytes, including the formation of tight junctions. It was previously shown that nanoparticles with decreasing sizes exhibit monotonically increasing permeability across Caco-2 cell monolayers, but few studies have focused on the ultrasmall (sub-10 nm diameter) size regime of nanoparticles. The studies that did focus on ultrasmall nanoparticles, essentially based on metals (e.g. gold) or metal oxides (e.g. iron oxide), showed that they indeed exhibit permeability through Caco-2 cells and mouse intestines. It is important to note that none of these studies were based on silica, however, as silica is typically expected to be unstable at the highly acidic environment of the stomach and is therefore considered not to be particularly suited to oral delivery, especially for organic ligand stabilized ultrasmall silica nanoparticles where dissolution rates or acid based chemical conversions are expected to be fast.

Past oral delivery studies of silica nanoparticles have shown that particles with diameters>20 nm are able to modulate the interactions between tight junction protein ZO-1 and actin cytoskeleton in Caco-2 cells. While this effectively “opens” tight junctions, it does not create large enough openings for the nanoparticles themselves to demonstrate permeability. Furthermore, it was suggested that the major complications that current silica nanoparticles face in oral delivery are poor dispersity, low permeability, and complex synthesis.

### SUMMARY OF THE DISCLOSURE

In an aspect, the present disclosure provides compositions and methods for oral delivery. In various examples, the methods comprise orally administering one or more composition(s), e.g., one or more composition(s) of the present disclosure, to an individual. A composition comprises a plurality of silica nanoparticles and/or aluminosilicate nanoparticles. In various examples, each nanoparticle independently comprises a silica or an aluminosilicate core and a plurality of polyethylene glycol (PEG) groups disposed (e.g., covalently bound by a linking group, or the like) on at least a portion of one or more nanoparticle surface(s). In various examples, nanoparticles have a longest linear dimension of about 20 nm or less (e.g., about 10 nm or less, such as, for example, about 7 nm or less). In various examples, at least a portion of the nanoparticles are delivered to the post-stomach portion of the individual's gastrointestinal tract. At least a portion of the nanoparticles (e.g., delivered nanoparticles, or the like) can pass through the mucus layer and permeate the epithelial lining of the gastrointestinal tract.

In various examples, nanoparticles of the present disclosure comprise various targeting, diagnostic, or therapeutic group(s), or the like, or any combination thereof, disposed (e.g., covalently bound by a linking group, or the like) on at least a portion of one or more nanoparticle surface(s) and/or the silica or aluminosilicate nanoparticle matrix. In various examples, at least a portion of the nanoparticles are in a form that retains at least a portion of one or more targeting, diagnostic, or therapeutic activit(ies), or the like, or any combination thereof, of the nanoparticles.

Composition(s) of the present disclosure can further comprise(s) one or more material(s) that render(s) a composition suitable for delivery of at least a portion of the nanoparticles to the post-stomach portion of the gastrointestinal tract of an individual to whom the composition has been orally administered and/or additive(s) that render the composition suitable for sustained nanoparticle release, delayed nanoparticle release, controlled nanoparticle release, time-dependent nanoparticle delivery, or any combination thereof. In various examples, the plurality of nanoparticles is disposed within the material(s) (e.g., a coating encasing the plurality of nanoparticles, or the like). In various examples, the material(s) is/are enteric and/or polymeric material(s) (e.g., pH-sensitive polymeric material(s)). In various examples, the additive(s) is/are pharmaceutically acceptable excipient(s). In various examples, composition(s) is/are in the form of a pill, a capsule, a tablet, a dragée, a bead, or a granule.

In an aspect, the present disclosure provides methods of targeting, diagnosing, treating, preventing, or the like, or any combination thereof, a current or potential disease, disease state, condition, disorder, side effect, or the like, or any combination thereof, in an individual. In various examples, the method comprises orally administering one or more composition(s) of the present disclosure to an individual in need of said targeting, diagnosing, treating, preventing, or the like, or any combination thereof. In various examples, the current or potential disease, disease state, condition, disorder, side effect, or the like, or any combination thereof, is chosen from infections, cancers, neurological conditions/diseases, neurodegenerative diseases, psychological conditions/diseases, inflammatory conditions/diseases, cardio-vascular diseases, and the like, and any combination thereof.

### BRIEF DESCRIPTION OF THE FIGURES

For a fuller understanding of the nature and objects of the disclosure, reference should be made to the following detailed description taken in conjunction with the accompanying figures herein.

FIGS. 1A-1B shows a schematic of the two major barriers to oral delivery. The first barrier is the mucosal lining that inhibits the free diffusion, e.g., of nanoparticles as disclosed herein (shown in expanded view in FIG. 1B), preventing them from reaching the epithelial lining. The second barrier is the epithelial lining itself, containing, e.g., tight cell junctions that restrict the direct passage of drugs, e.g., in the form of nanoparticles.

FIGS. 2A-2B show synthesis workflows of the two nanoparticle synthesis routes used in this study. (FIG. 2A) Smaller nanoparticles with hydrodynamic diameters below 30 nm were synthesized by co-condensing TMOS and dye-silane in aqueous solution followed by PEGylation, with optional TEOS based shell addition(s) until the desired size was reached and terminated by PEGylation. (FIG. 2B) Larger nanoparticles with hydrodynamic diameters above 30 nm were formed by adding TEOS and dye-silane based shells to silica seeds grown in an l-arginine/water solution until the desired size was achieved and terminated by PEGylation.

FIGS. 3A-3G show nanoparticle characterization. (FIG. 3A) Confocal FCS of all nanoparticles with larger nanoparticles exhibiting slower diffusion than smaller ones. (FIG. 3B) UV/Vis absorbance spectra of all nanoparticles normalized to dye absorption peak. With increasing particle sizes, the increasing scattering background at lower wavelength can clearly be discerned. Nanoparticles with diameters of 18.9 nm and 24.7 nm encapsulate ATTO647N, while the rest were synthesized with Cy5. (FIGS. 3C-3G).TEM images of 6.3 nm, 10.0 nm, 18.9 nm, 24.7 nm, and 33.9 nm, respectively. TEM images of 47.1 nm nanoparticles are not shown here.

FIGS. 4A-4F show confocal FCS raw data for each nanoparticle size. (FIG. 4A) 6.3 nm, (FIG. 4B) 10.0 nm, (FIG. 4C) 18.9 nm, (FIG. 4D) 24.7 nm, (FIG. 4E) 33.9 nm, (FIG. 4F) 47.1 nm along with their corresponding fits to equation (2) disclosed herein.

FIGS. 5A-5D show nanoparticle diffusion in different media characterized via different FCS setups. (FIG. 5A) Nanoparticles diffusing in water can conveniently be studied using confocal FCS. (FIG. 5B) Confocal FCS cannot be used for analyzing nanoparticles diffusing in mucus due to the high scattering background of this medium. (FIG. 5C) Imaging FCS using a TIRF setup circumvents a high scattering background but is problematic when a subset of nanoparticles adheres to the glass substrate. (FIG. 5D) Imaging FCS with a leaky TIRF setup enables detection signal primarily from particles diffusing above the glass substrate while simultaneously limiting the effects of scattering.

FIGS. 6A-6D show results for nanoparticle diffusion in mucus obtained from leaky TIRF-based imaging FCS. (FIG. 6A) Autocorrelation functions and their fits of 6.3 nm particles diffusing in mucus. (FIG. 6B) Autocorrelation functions and their fits of 33.9 nm particles diffusing in mucus. Insets in FIGS. 6A-6B show individual correlation curves with fits. (FIG. 6C) Average autocorrelation functions for all nanoparticle sizes demonstrating the dependence of diffusion time on nanoparticle size. (FIG. 6D) Cumulative distribution functions demonstrating the change in diffusion coefficient in mucus as nanoparticle size changes.

FIGS. 7A-7C show a comparison of nanoparticle diffusion in water vs mucus. The diffusion coefficient of nanoparticles decreases monotonically as size increases in both (FIG. 7A) water (D_{water}) and (FIG. 7B) mucus (D_{mucus}). (FIG. 7C) Taking the ratio of D_{mucus}/D_{water} demonstrates that nanoparticles diffuse slower in mucus than in water and that particles<20 nm hydrodynamic diameter appear to follow a different trend than those >20 nm.

FIGS. 8A-8C show caco-2 permeability assay results for nanoparticles of different sizes. (FIG. 8A) Schematic of the Transwell assay used to study the permeability of nanoparticles through Caco-2 monolayer. (FIG. 8B) Lucifer Yellow assay demonstrating that all Caco-2 cell monolayers used had well-formed tight junctions. Cell Groups 1, 2, and 3 were used for the 6.3 nm, 24.7 nm, and 33.9 nm nanoparticles shown in FIG. 8C, respectively. (FIG. 8C) Apparent permeability (Pₐₚₚ) of nanoparticles through the Caco-2 monolayers, which decreased as nanoparticle size increased, with the 33.9 nm nanoparticle permeability being undetected. * p<0.05 as determined via a student's t-test with n=3.

FIGS. 9A-9B show: (FIG. 9A) Confocal FCS of 5 nm and 26 nm nanoparticles encapsulating Cy5 and their fits. (FIG. 9A) Replicate experiment of nanoparticle permeability through Caco-2 monolayers demonstrating similar results to those presented herein. * p<0.05 as determined via a student's t-test with n=3.

FIGS. 10A-10F show fluorescence laser scanning confocal microscopy of fixed Caco-2 cells. Membranes were exposed to either (FIGS. 10A-10C) 6.3 nm or (FIGS. 10D-10F) 24.7 nm silica nanoparticles (Cy5). Tight junction protein ZO-1 was immunostained using Alexa Fluor 488-antibody conjugates.

FIGS. 11A-11F show replicate fluorescence laser scanning confocal microscopy of fixed Caco-2 cells demonstrating similar results to those presented in the main text. Membranes were exposed to either (FIGS. 11A-11C) 6.3 nm or (FIGS. 11D-11F) 24.7 nm silica nanoparticles (Cy5). Tight junction protein ZO-1 was immunostained using Alexa Fluor 488-antibody conjugates.

FIGS. 12A-12D show nanoparticle stability in artificial gastric juice and nanoparticle pill formulation. (FIG. 12A) Nanoparticle size versus time incubated in either artificial gastric juice, pH 1.5 HCl, or pH 13 NH₃. (FIG. 12B) Photo of a pill formulation inside a 1.5 mL microcentrifuge tube. (FIG. 12C) Fluorescence image of pill formulation in FIG. 12B using an IVIS imaging system with 660 nm emission filter. (FIG. 12D) Overlay of pill photo and pill fluorescence image.

### DETAILED DESCRIPTION OF THE DISCLOSURE

Although subject matter of the present disclosure is described in terms of certain embodiments and examples, other embodiments and examples, including embodiments and examples that do not provide all of the benefits and features set forth herein, are also within the scope of this disclosure. For example, various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives reasonably expected to perform in substantially the same manner or the same manner as the listed alternatives. As used herein, unless otherwise stated, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about”, it will be understood that the particular value forms a further disclosure. For example, if the value “about 10” is disclosed, then “10” is also disclosed.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent radicals and multivalent radicals, such as, for example, divalent radicals, trivalent radicals, and the like). Illustrative examples of groups include:

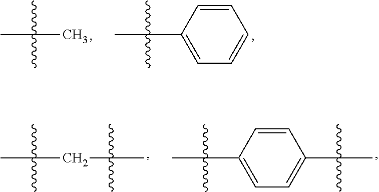

and the like.

As used herein, unless otherwise stated, the term “derived” refers to formation of a group by reaction of a native functional group of a compound (e.g., formation of a group via reaction of an amine of a compound and a carboxylic acid to form a group or the like), chemical modification of a compound to introduce a new chemically reactive group on the compound that is reacted to form a group, or the like.

The present disclosure provides compositions comprising nanoparticles for oral delivery. The present disclosure also provides methods of oral delivery of nanoparticles and treatment methods.

In an aspect, the present disclosure provides compositions. A composition comprises a plurality of nanoparticles. The nanoparticles may be silica nanoparticles, aluminosilicate nanoparticles, or a combination thereof. The nanoparticles may be surface functionalized with polyethylene glycol groups (e.g., PEGylated nanoparticles). Non-limiting examples of compositions are disclosed herein.

In various examples, a composition (e.g., an oral delivery composition) comprises a plurality of silica nanoparticles and/or aluminosilicate nanoparticles. In various examples, a composition (e.g., an oral delivery composition, such as, for example, an oral delivery composition comprising one or more nanoparticle(s), one or more nanoparticle(s) and enteric material(s), or the like) is suitable for delivery of the silica nanoparticles and/or aluminosilicate nanoparticles to the post-stomach portion of the gastrointestinal tract (e.g., large intestine, small intestine, colon, ileum, or the like, or a combination thereof) of an individual to whom the composition has been orally administered. In various examples, each nanoparticle independently comprises a silica core or aluminosilicate core. In various examples, an aluminosilicate core comprises an Al/Si atomic ratio of 0.01 to 30%, including all 0.01 at. % values and ranges therebetween.

A nanoparticle may be a core nanoparticle (e.g., a nanoparticle comprising an inner (“core”) region comprising a core composition and/or structure, such as, for example, an inorganic core nanoparticle, or the like), a core-shell nanoparticle (e.g., a nanoparticle comprising: an inner (“core”) region, such as, for example, an inorganic core, or the like, comprising a core composition and/or structure; and one or more outer (“shell”) region(s) disposed on at least a portion or all of the outer surface(s) of the inner (“core”) region, including one or more same or different shell composition(s) and/or structure(s), such as, for example, one or more inorganic and/or organic shell(s), or the like). In various examples, a nanoparticle is a PEGylated nanoparticle (e.g., a core-shell nanoparticle comprising: an inner (“core”) region comprising a core composition and/or structure, such as, for example, an inorganic core nanoparticle, or the like; and one or more outer (“PEGylated shell(s)”) comprising a shell composition and/or structure comprising polyethylene glycol groups). Any of these nanoparticles can be silica nanoparticles or aluminosilicate nanoparticles.

A composition can comprise one or more type(s) of nanoparticles (e.g., having different average size and/or one or more different compositional feature(s)). For example, a composition comprises a plurality of core and/or core-shell nanoparticles (e.g., silica core nanoparticles, silica core-shell nanoparticles, aluminosilicate core nanoparticles, aluminosilicate core-shell nanoparticles, or the like). Any of the nanoparticles may be surface functionalized with one or more type(s), size(s), surface densit(ies), or the like, of polyethylene glycol groups (e.g., polyethylene glycol groups, functionalized (e.g., functionalized with one or more ligand(s) and/or reactive group(s)) polyethylene glycol groups, or a combination thereof). In various examples, one or more of the nanoparticle(s) has/have at least one structural and/or compositional feature (e.g., core structure (e.g., core, core-shell, or the like), core composition (e.g., silicon core, aluminosilicate core, Al/Si ratio, or the like), encapsulated additive concentration (e.g., dye or the like), surface group composition (e.g., type (e.g., PEG group, targeting group, therapeutic group, diagnostic group, or the like), size, surface density, or the like)) different that one or more or all of the other nanoparticles. Nanoparticles can be made by a method of the present disclosure.

Suitable silica nanoparticles and aluminosilicate nanoparticles are known in the art. In various examples, the silica nanoparticles and aluminosilicate nanoparticles are Cornell dots (e.g., C dots or C′ dots), a portion or all of which may be PEGylated. In various examples, a silica nanoparticle and/or aluminosilicate nanoparticle is a silica nanoring and/or aluminosilicate nanoring, silica nanocage and/or aluminosilicate nanocage, or the like, or any combination thereof, a portion or all of which may be PEGylated. Non-limiting examples of nanoparticles are described in U.S. Published Patent Application Nos. 20130177934 (Aminated Mesoporous Silica Nanoparticles, Methods of Making Same, and Uses Thereof, filed Dec. 17, 2012), 20160018404 (Multilayer fluorescent nanoparticles and methods of making and using same, filed Feb. 20, 2014), 20180133346 (Ultrasmall nanoparticles and methods of making and using same, filed May 4, 2016), 20190282712 (Inhibitor-functionalized ultrasmall nanoparticles and methods thereof, filed Nov. 29, 2017), 20190351077 (Cyclic peptides with enhanced nerve-binding selectively, nanoparticles bound with said cyclic peptides, and use of same for real-time in vivo nerve tissue imaging, filed May 17, 2019), 20200101180 (Ultrasmall nanoparticles labeled with zirconium-89 and methods thereof, filed May 27, 2018), 20200179538 (Functionalized nanoparticles and methods of making and using same, filed May 21, 2018), 20200316219 (Methods of treatment using ultrasmall nanoparticles to induce cell death of nutrient-deprived cancer cells via ferroptosis, filed Jun. 16, 2020), 20200353096 (Nanoparticle Immunoconjugates, filed Dec. 19, 2019), 20210048414 (Ultrasmall nanoparticles and methods of making, using and analyzing same, filed May 2, 2019), and International Publication No. WO 2017/189961 (Compositions and methods for targeted particle penetration, distribution, and response in malignant brain tumors, filed Apr. 28, 2017), International Publication No. WO 2020/214741 (Functionalized silica nanorings, methods of making same, and uses thereof, filed Apr. 15, 2020) and U.S. Pat. No. 8,298,677 (Fluorescent silica-based nanoparticles, filed Nov. 26, 2003), U.S. Pat. No. 8,961,825 (Fluorescent silica nanoparticles through silica densification, filed Apr. 15, 2010), U.S. Pat. No. 8,084,001 (Photoluminescent silica-based sensors and methods of use, filed May 2, 2005), U.S. Pat. No. 10,111,963 (Nanoparticle Drug Conjugates, filed May 27, 2015), U.S. Pat. No. 10,548,997 (Fluorescent silica-based nanoparticles, filed Mar. 1, 2017), U.S. Pat. No. 10,548,998 (Multimodal silica-based nanoparticles, filed Jun. 15, 2018), U.S. Pat. No. 10,732,115 (Mesoporous oxide nanoparticles and methods of making and using same, filed Jun. 6, 2013), U.S. Pat. No. 10,732, 115 (Mesoporous oxide nanoparticles and methods of making and using same, filed Jun. 6, 2013), the disclosure(s) of which with regard to silica nanoparticles and/or aluminosilicate nanoparticles, uses of same, and methods of making same are incorporated herein by reference. In various examples, a composition comprises or the nanoparticles comprise (or are) those described in and/or made by a method disclosed in one or more of these U.S. Patent(s) and/or U.S. Published Patent Application(s).

In various examples, the silica nanoparticles and/or the aluminosilicate nanoparticles in a composition have a variety of sizes (e.g., a longest linear dimension, which may be a diameter, or the like (e.g., a hydrodynamic diameter, a TEM diameter, or the like)) and/or size distributions. In various examples, the nanoparticles, independently, have a size (e.g., a core size, a core-shell size, a size including PEG groups, a size excluding PEG groups, a size for each nanoparticle independently, an average size for a plurality of nanoparticles, or the like, or any combination thereof) of about 20 nm or less (e.g., about 10 nm or less, or about 7 nm or less). In various examples, the nanoparticles, independently, have a size (e.g., a core size, a core-shell size, a size including PEG groups, a size excluding

PEG groups, a size for each nanoparticle independently, an average size for a plurality of nanoparticles, or the like, or any combination thereof) of from about 3 nm to about 20 nm, including all 0.1 nm values and ranges therebetween (e.g., from about 3 nm to about 19.5 nm, from about 5 nm to about 15 nm, or about 10 nm, including all 0.1 nm values and ranges therebetween). In various examples, the nanoparticles have a size (e.g., a core size, a core-shell size, a size including PEG groups, a size excluding PEG groups, a size for each nanoparticle independently, an average size for a plurality of nanoparticles, or the like, or any combination thereof) of about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, or about 20 nm. In various examples, at least about 90%, about 95%, about 96%, about 97%, about 98%, about 99%, about 99.5% about 99.9%, or about 100% of the nanoparticles have a size (e.g., a core size, a core-shell size, a size including PEG groups, a size excluding PEG groups, a size for each nanoparticle independently, an average size for a plurality of nanoparticles, or the like, or any combination thereof) of from about 3 nm to about 20 nm (e.g., from about 2 nm to about 20 nm), including all 0.1 nm values and ranges therebetween. In various examples, at least a portion of or all of the nanoparticles (e.g., silica nanoparticles and/or alumina nanoparticles) in a composition have size and/or size distribution described in this paragraph.

For the exemplary particle size distributions of nanoparticles described herein, the composition may not be subjected to any particle-size discriminating (particle size selection/removal) processes (e.g., filtration, dialysis, chromatography (e.g., GPC), centrifugation, etc.). The nanoparticles may be as synthesized and not have any post-synthesis processing/treatment. For the exemplary particle size distributions described herein, the composition may be subjected to one or more purification step(s) in which larger particle aggregates, smaller chemical reagents, or the like, or a combination thereof, are separated from nanoparticles.

The nanoparticles may have a narrow particle size distribution. In various examples, the particle size distribution of the nanoparticles, not including extraneous materials, such as, for example, aggregates, unreacted reagents, dust particles/aggregates, is +/−5, 10, 15, or 20% of the average particle size (e.g., a longest linear dimension).

Particle size and distribution (e.g., a core size/distribution, a core-shell size/distribution, a size/distribution including PEG groups, a size/distribution excluding PEG groups, a size for each nanoparticle independently, an average size/distribution for a plurality of nanoparticles, or the like, or any combination thereof) can be determined by methods known in the art. In various examples, a particle size is determined by chromatography (e.g., gel permeation chromatography or the like), spectroscopy (e.g., dynamic light scattering (DLS), fluorescence correlation spectroscopy (FCS), or the like), electron microscopy (e.g., transmission electron microscopy (TEM), scanning electron microscopy (SEM), or the like) or the like. DLS contains systematic deviation and, therefore, the DLS size distribution may not correlate with the particle size distribution determined by TEM or GPC.

In various examples, each nanoparticle comprises a plurality of polyethylene glycol (PEG) groups disposed on (e.g., covalently bound to, or the like) at least a portion of or all of a surface or a portion of or all of the surfaces of the nanoparticle. These PEG groups may be referred to, in the alternative, as an outer (“PEGylated shell”). In various examples, nanoparticles comprising a plurality of PEG groups, independently and/or on average, comprise a size (e.g., a longest linear dimension (which may be a diameter, such as, for example, a hydrodynamic diameter, a TEM diameter, or the like)) of 20 nm or less (e.g., less than 20 nm, less than 15 nm, or less than 10 nm, or less than 7 nm).

In various examples, a nanoparticle has a PEG layer of various dimensions. In various examples, the chain length of the PEG groups (i.e., the molecular weight of the PEG group), individually or on average, is tuned from about 2 EO groups to about 20 EO groups, including all integer number of EO groups and ranges therebetween (e.g., from about 5 EO groups to about 10 EO groups, or from about 6 EO groups to about 9 EO groups, including all integer number of EO groups and ranges therebetween) (e.g., about 2, about 3, about 4, about 5, about 6, about 7, about 8, about 9, about 10, about 11, about 12, about 13, about 14, about 15, about 16, about 17, about 18, about 19, or about 20 EO group(s)). In various examples, PEG chain length is selected to tune the thickness of a PEG layer surrounding the nanoparticle and the pharmaceutical kinetics profiles of PEGylated nanoparticles. In various examples, a PEG layer comprises various PEG group surface densities on a nanoparticle. In various examples, a nanoparticle comprises a PEG group surface density of from about 1.2 PEG groups/ nm2 to 2.2 PEG groups/nm2, including all 0.01 PEG groups/nm2 and ranges therebetween. In various examples, a PEG group is covalently bound to at least a portion of or all of a surface or a portion of or all of the surfaces of a nanoparticle. In various examples, a PEG group is a portion of a larger/more complex group disposed on (e.g., covalently bound to or the like) a surface or a portion of or all of the surfaces of a nanoparticle (e.g., via a linking group). In various examples, at least a portion of or all of the PEG groups comprise one or more ligand(s) (such PEG groups are also referred to as functionalized PEG groups). In various examples, at least a portion or all of the PEG groups comprise one or more targeting group(s), one or more diagnosing group(s), one or more therapeutic group(s), or any combination thereof.

One or more or all of the silica nanoparticles and/or aluminosilicate nanoparticles may carry ligands. In various examples, the ligands are chosen from targeting groups, therapeutic groups (e.g., a drug group or the like), and diagnostic groups, and the like, and any combination thereof, any of which may be referred to as a functional group, a moiety, a ligand, or the like. In various examples, a ligand is disposed on (e.g., covalently bound to, or the like) one or more surfaces (e.g., an exterior surface, a pore surface, or the like, or any combination thereof) or encapsulated by (e.g., covalently bound to) the silica matrix or aluminosilicate matrix of one or more or all of nanoparticle(s). In various examples, a ligand, such as, for example, a dye group or the like, is at least partially or completely encapsulated by and/or covalently bound the silica and/or aluminosilicate matrix of a nanoparticle. In various examples, a ligand is covalently bound to a nanoparticle via a linking group (e.g., a fluorescent dye linked to a silane and covalently bonded to the silica nanoparticles and/or aluminosilicate matrix via the silane). In various examples, a targeting group, a therapeutic group (e.g., a drug or the like), a diagnostic group, or the like, is a portion of a larger/more complex group (e.g., a functionalized PEG group) covalently bound to a surface or a portion of or all of the surfaces of a nanoparticle or nanoparticles (e.g., via a linking group).

Methods of forming covalently bonded groups are known in the art. In various, examples, one or more group(s) are formed by post-PEGylation surface modification by insertion (PPSMI).

In various examples, an individual nanoparticle comprises from about 1 to about 5, including all integer values and ranges therebetween, (e.g., about 1, about 2, about 3, about 4, or about 5) types of different ligands (which may be groups) within (e.g., at least partially or completely encapsulated by, covalently bound to the silica matrix or the aluminosilicate matrix, or the like) the nanoparticle matrix and/or disposed on the nanoparticle surface. Without intending to be bound by any particular theory, it is considered that the PEG chain length may be used to tune the accessibility of the ligand groups on the surface of the PEG layer of the particles resulting in varying binding and targeting performance.

A nanoparticle may comprise various targeting groups. A targeting group (e.g., an affinity ligand or the like) facilitates targeted delivery of a nanoparticle or nanoparticles. A targeting group may be formed from (derived from) a targeting molecule, biological material, or the like. In various examples, a targeting group derived from a targeting molecule, biological material, or the like has substantially the same properties (e.g., activity, which may be biological activity or the like) as the targeting molecule, biological material, or the like from which it is derived. A targeting group (e.g., an affinity ligand or the like) may be conjugated to the nanoparticle (e.g., to a surface of the nanoparticle) to allow targeted delivery of the nanoparticles. In various examples, a nanoparticle is conjugated to a ligand which is capable of binding to a cellular component (e.g., on the cell membrane or in the intracellular compartment) associated with a specific cell type. In various examples, the targeted molecule is a tumor marker or a molecule in a signaling pathway or the like. In various examples, the ligand has specific binding affinity to certain cell types, such as, for example, tumor cells and the like. In various examples, the ligand is used for guiding the nanoparticles to specific areas, such as, for example, liver, spleen, brain, or the like.

Non-limiting examples of targeting groups include groups with targeting ability (e.g., antibody fragments, aptamers, proteins/peptides (natural, truncated, or synthetic), nucleic acids such as, for example, DNA and RNA, and the like). Non-limiting examples of targeting groups include linear and cyclic peptides (e.g., integrin-targeting cyclic(arginine-glycine-aspartic acid-D-tyrosine-cysteine) peptides, c(RGDyC), and the like), antibody fragments, various DNA and RNA segments (e.g. siRNA). Other non-limiting examples of targeting groups include cancer-targeting peptides, and the like, and any combination thereof.

A nanoparticle may comprise various diagnostic groups. In various examples, a diagnostic group provides diagnostic information about an individual. A diagnostic group may be formed from (derived from) a molecule, an atom, a biological material, or the like. Non-limiting examples of diagnostic groups include groups having absorption/emission behavior such as, for example, fluorescence and phosphorescence, which in various examples is used for imaging, sensing functionality (e.g., pH sensing, ion sensing, oxygen sensing, biomolecules sensing, temperature sensing, and the like), or the like. In various examples, a diagnostic group is chosen from dye groups, sensor groups, radioisotopes, and the like, and any combination thereof. In various examples, imaging is used to determine the location of the nanoparticles in an individual.

In various examples, a nanoparticle comprises various dyes (e.g., groups formed from various dyes). In various examples, at least a portion of the nanoparticles have a dye or combination of dyes (e.g., a NIR dye) encapsulated therein. In various examples, the dye groups (which may be formed from dye molecules) are covalently bound to the nanoparticles (e.g., where the dye groups are at least partially or completely encapsulated by, covalently bound to the silica matrix or the aluminosilicate matrix, or the like and/or disposed on the nanoparticle surface). In various examples, the dyes are organic dyes. In an example, a dye does not comprise a metal atom. Non-limiting examples of dyes include fluorescent dyes (e.g., near infrared (NIR) dyes and the like), phosphorescent dyes, non-fluorescent dyes (e.g., non-fluorescent dyes exhibiting less than 1% fluorescence quantum yield), fluorescent proteins (e.g., EBFP2 (variant of blue fluorescent protein), mCFP (Cyan fluorescent protein), GFP (green fluorescent protein), mCherry (variant of red fluorescent protein), iRFP720 (Near Infra-Red fluorescent protein)), and the like, and groups derived therefrom. In various examples, a dye absorbs in the UV-visible portion of the electromagnetic spectrum. In various examples, a dye has an excitation and/or emission in the near-infrared portion of the electromagnetic spectrum (e.g., 650-900 nm).

Non-limiting examples of organic dyes include cyanine dyes (e.g., Cy5®, Cy3®, Cy5.5®, Cy7®, and the like), carborhodamine dyes (e.g., ATTO 647N (available from ATTO-TEC and Sigma Aldrich®), BODIPY dyes (e.g., BODIPY 650/665 and the like), xanthene dyes (e.g., fluorescein dyes such as, for example, fluorescein isothiocyanate (FITC), Rose Bengal, and the like), eosins (e.g. Eosin Y and the like), and rhodamines (e.g. TAMRA, tetramethylrhodamine (TMR), TRITC, DyLight® 633, Alexa 633, HiLyte 594, and the like), Dyomics® DY800, Dyomics® DY782 and IRDye® 800CW, and the like, and groups derived therefrom.

In various examples, a diagnostic group is a sensor group. A nanoparticle may comprise various sensor groups. Non-limiting examples of sensor groups include pH sensing groups, ion sensing groups, oxygen sensing groups, biomolecule sensing groups, temperature sensing groups, and the like. Examples of suitable sensing compounds/groups are known in the art.

In various examples, a group (such as, for example, a therapeutic group, a diagnostic group, or the like) comprises a radioisotope. In various examples, a radioisotope is a diagnostic agent and/or a therapeutic agent. For example, a radioisotope, such as for example, ¹²⁴I, is used for positron emission tomography (PET), and the like. Non-limiting examples of radioisotopes include ¹²⁴, ¹³¹I, 89Zr, 64Cu, as well as radiotherapeutic isotopes, such as, for example, ²²⁵Ac, ¹⁷⁷Lu, and the like. A radioisotope may be chelated to a chelating group.

A nanoparticle may comprise various chelator groups. Non-limiting examples of chelator groups include desferoxamine (DFO), 1,4,7,10-Tetraazacyclododecane-1,4,7,10-tetraacetic acid (DOTA), 1,4,7-triazacyclononane-1,4,7-triacetic acid (NOTA), ethylenediaminetetraacetic acid (EDTA), diethylenetriaminepentaacetic acid (DTPA), porphyrins, and the like, and groups derived therefrom. A chelator group may comprise a radioisotope. Non-limiting examples of radioisotopes are described herein and suitable examples of radioisotopes are known in the art.

A nanoparticle may comprise various therapeutic groups. As referred to herein, unless otherwise stated, a therapeutic group is defined as any molecule, atom, or the like, or any combination thereof, with therapeutic ability (e.g., drugs (which may be small molecule drugs and the like), nucleic acids, biological materials, radioisotopes, and the like, and any combination thereof). In various examples, a therapeutic group is formed from (derived from) a molecule, atom, or the like with therapeutic ability. In various examples, a therapeutic group releases a therapeutic agent (which may be the native form or an active form of a drug, nucleic acid, or the like) from a nanoparticle having substantially all (e.g., at least 90%, at least 95%, or at least 99% of the parent drug's activity) or all of the native (e.g., unconjugated form of the drug, nucleic acid, or the like) drug's, nucleic acid's, or the like's activity. In various examples, a therapeutic group is formed from a drug (which may be a small molecule drug), a nucleic acid, or the like.

A group may have both imaging and therapeutic functionality. In various examples, a group having both imaging and therapeutic functionality is formed from a compound or radioisotope exhibiting imaging and therapeutic functionality by derivatization of the compound and/or radioisotope using conjugation chemistry and reactions known in the art.

Non-limiting examples of therapeutic agents include, but are not limited to, chemotherapeutic agents, small molecule inhibitors, cytotoxic drugs, antibiotics, antifungal agents, antiparasitic agents, antiviral agents, anti-inflammatory agents, neurological agents, psychotherapy agents, groups comprising one or more radiotherapeutic isotope(s) (such as, for example, ²²⁵Ac, ¹⁷⁷Lu, and the like), and the like, and any combination thereof. Any of these agents may be drugs (e.g., drugs, which may be small molecule drugs and the like, nucleic acids, biological materials, radioisotopes, and the like). In various examples, a therapeutic group is formed from (derived from) one of these therapeutic agents.

In various examples, a therapeutic group is a drug group. A variety of drugs (e.g., small molecule drugs and the like) can be used to form a drug group. In various examples, a drug disposed on is conjugated to a surface of a nanoparticle. Drugs can be conjugated to a surface of a nanoparticle by methods known in the art. A drug group may release a drug from a nanoparticle having substantially all (e.g., at least 90%, at least 95%, or at least 99% of the parent drug's activity) or all of the parent drug's activity.

In various examples, a therapeutic group (e.g., a drug group, such as, for example, a drug-linker conjugate group, where the linker group is capable of being specifically cleaved by enzyme or acid condition in tumor for drug release, is disposed (e.g., covalently bonded to) a surface of a nanoparticle (e.g., attached to a functional ligand on a surface of a nanoparticle) for drug delivery. In various examples, drug-linker-thiol conjugates are attached to maleimido-PEG-particles through thiol-maleimido conjugation reaction post the synthesis of maleimido-PEG-particles.

It may be desirable to form a drug group from a hydrophobic drug. In various examples, a drug group is a hydrophobic drug group. Therapeutic groups may be formed from (e.g., derived from) therapeutic agents (e.g., drugs, which may be small molecule drugs, such as, for example, small molecule inhibitors, cytotoxic drugs, and the like, and the like), nucleic acids, biological materials, radioisotopes, and the like), and the like, that are not considered amenable to oral administration.

In various examples, at least a portion of the silica nanoparticles and/or the aluminosilicate nanoparticles are capable of passing through the mucus layer and permeate the epithelial lining of the post-stomach portion of the gastrointestinal tract of an individual to whom the composition(s) is/are orally administered. In various examples, the silica nanoparticles and/or the aluminosilicate nanoparticles exhibit one or more or all of the following: a diffusion coefficient through the mucus layer (D_{mucus}) of from about 5 μm²/s to about 35 μm²/s, including all 0.1 μm²/s values and ranges therebetween; a diffusion coefficient through water (D_{water}) of from about 20 μm²/s to about 70 μm²/s, including all 0.1 μm²/s values and ranges therebetween; or a ratio of the diffusion coefficients through the mucus layer and water (D_{mucus}/D_{water}) of from about 0.1 to about 0.5, including all 0.01 diffusion coefficient ratio values and ranges therebetween.

In various examples, a composition further comprises one or more material(s) that render the composition suitable for delivery of the nanoparticles (e.g., silica nanoparticles and/or aluminosilicate nanoparticles) to the post-stomach portion of the gastrointestinal tract (e.g., large intestine, small intestine, colon, ileum, or the like, or a combination thereof) of an individual to whom the composition has been orally administered (e.g., enteric material and the like). In various examples, a plurality of (e.g., substantially all or all of) the nanoparticles (e.g., silica nanoparticles and/or aluminosilicate nanoparticles) is disposed within (e.g., are sequestered by, coated by, or the like) the one or more material(s). In various examples, substantially all or all of the surfaces of a plurality of (e.g., substantially all or all of) the silica nanoparticles and/or aluminosilicate nanoparticles is coated by the one or more material(s). In various examples, the material(s) are present as one or more layer(s). In various examples, the individual layers are the same or one or more of the layers is/are different in terms of at least one structural and/or compositional feature (e.g., material(s), thickness, or the like). In various examples, the material(s), in total, provide a coating thickness (e.g., of one or more layers) of about 20 nm to about 350 micron, including all 0.1 micron values and ranges therebetween, on the nanoparticles. In various examples, the material(s) that render a composition suitable for delivery of the silica nanoparticles and/or aluminosilicate nanoparticles to the post-stomach portion of the gastrointestinal tract (e.g., large intestine, small intestine, colon, or the like, or a combination thereof) of an individual, which may be enteric material(s), are the outermost material(s) (e.g., the outermost layer of materials) of the compositions.

In various examples, a variety of materials that render a composition suitable for delivery of the nanoparticles (e.g., silica nanoparticles and/or aluminosilicate nanoparticles) to the post-stomach portion of the gastrointestinal tract (e.g., large intestine, small intestine, colon, or the like, or a combination thereof) of an individual are used in the compositions. In various examples, the material(s) remain at least partially, substantially, or completely intact until the composition (e.g., at least a portion of or all of the nanoparticles) passes through the stomach or until the composition reaches the post-stomach portion of the gastrointestinal tract (e.g., large intestine, small intestine, colon, or the like, or a combination thereof) of the individual.

In various examples, at least a portion or all of the materials are enteric materials. In various examples, a material that renders a composition suitable for delivery of the nanoparticles (e.g., silica nanoparticles and/or aluminosilicate nanoparticles) to the post-stomach portion of the gastrointestinal tract (e.g., large intestine, small intestine, colon, or the like, or a combination thereof) of an individual (e.g., an enteric material or the like) is non-toxic, soluble in the post-stomach portion of the gastrointestinal tract (e.g., large intestine, small intestine, colon, ileum, or the like, or a combination thereof) of an individual, insoluble or substantially insoluble in gastric juice, or any combination thereof. In various examples,

In various examples, a composition is a pH sensitive composition. In various examples, a material is a pH sensitive material. In various examples, a material (e.g., an enteric material or the like) is ionizable at a pH of from about 5 to about 7, including all 0.1 pH values and ranges therebetween.

Suitable materials are known in the art. Non-limiting examples of materials suitable for delivery of the silica nanoparticles and/or aluminosilicate nanoparticles to the post-stomach portion of the gastrointestinal tract (e.g., large intestine, small intestine, colon, ileum, or the like, or a combination thereof) of an individual (e.g., enteric materials and the like) are described in U.S. Pat. Nos. 8,535,718, 9,211,261, 9,592,248, 9,890,141, 10,005,784, and 10,336,774, the disclosure(s) of which with regard to enteric materials or materials suitable for delivery of the silica nanoparticles and/or aluminosilicate nanoparticles to the post-stomach portion of the gastrointestinal tract (e.g., large intestine, small intestine, colon, ileum, or the like, or a combination thereof) of an individual are incorporated herein by reference. In various examples, the enteric material material(s) are one or more of those described in one or more of these U.S. Patent(s).

Non-limiting examples of materials, which may be enteric materials, include polymeric materials. Non-limiting examples of polymeric materials include pH sensitive polymers, and the like, and any combination thereof. Non-limiting examples of pH-sensitive polymers include polyacrylamides, phthalate derivatives such as acid phthalate of carbohydrates, amylose acetate phthalate, cellulose acetate phthalate (CAP), other cellulose ester phthalates, cellulose ether phthalates, hydroxypropylcellulose phthalate (HPCP), hydroxypropyl ethylcellulose phthalate (HPECP), hydroxypropyl methylcellulose phthalate (HPMCP), HPMCAS, methylcellulose phthalate (MCP), carboxymethylethyl cellulose (CMEC), polyvinyl acetate phthalate (PVAcP), polyvinyl acetate hydrogen phthalate, sodium CAP, starch acid phthalate, cellulose acetate trimellitate (CAT), styrene-maleic acid dibutyl phthalate copolymer, styrene-maleic acid/polyvinylacetate phthalate copolymer, styrene and maleic acid copolymers, polyacrylic acid derivatives such as, for example, acrylic acid and acrylic ester copolymers, polymethacrylic acid and esters thereof, polyacrylic and methacrylic acid copolymers, shellac and copolymers of vinyl acetate and crotonic acid, and the like and any combination thereof. Other non-limiting examples of pH-sensitive polymers include shellac (trade name EmCoat 120 N, Marcoat 125); cellulose acetate phthalate (trade name aquacoat CPDR, Sepifilm™ LP, Klucel®, Aquacoat® ECD, and Metolose®); polyvinylacetate phthalate (trade name Sureteric®); and methacrylic acid (trade name Eudragit®), and the like and any combination thereof. Non-limiting examples of enteric materials include cellulose acetate phthalate, polyvinyl acetate phthalate, methacrylic acid-methacrylic acid ester copolymers, carboxymethyl ethylcellulose, and hydroxypropyl methylcellulose acetate succinates, and the like, and any combination thereof.

In various examples, at least a portion of the material(s) are one or more additional material(s). In various examples, the additional material(s) is/are chosen from sustained release materials, delayed release materials, controlled release materials, time-dependent delivery materials, or the like, or any combination thereof. An additional material may be suitable for delivery of the silica nanoparticles and/or aluminosilicate nanoparticles to the post-stomach portion of the gastrointestinal tract (e.g., large intestine, small intestine, colon, ileum, or the like, or a combination thereof) of an individual. At least a portion of, substantially all, or all of the additional material(s) may remain at least partially, substantially, or completely intact until the composition passes through the stomach or until the composition reaches the post-stomach portion of the gastrointestinal tract (e.g., large intestine, small intestine, colon, or the like, or a combination thereof) of the individual. In various examples, a composition comprising one or more additional material(s) exhibits sustained nanoparticle release, delayed nanoparticle release, controlled nanoparticle release, time-dependent nanoparticle delivery, or the like.

Examples of materials (which may be referred to as additional materials) that provide sustained nanoparticle release, delayed nanoparticle release, controlled nanoparticle release, time-dependent nanoparticle delivery compositions are known in the art. Non-limiting examples of additional materials are described in U.S. Pat. Nos. 8,535,718, 9,211,261, 9,592,248, 9,890, 141, 10,005,784, 10,336,774, the disclosure(s) of which with regard to additional materials (e.g., materials that provide sustained release, delayed release, controlled release, time-dependent, or the like) are incorporated herein by reference. In various examples, the additional material(s) are those described in one or more of these U.S. Patent(s) and/or U.S. Published Patent Application(s).

In various examples, a composition comprises additional component(s) (which may also be referred to as additive(s)). An additive may be a functional additive or non-functional additive. Non-limiting examples of additives include excipients (such as, for example, fillers, diluents, plasticizers, emulsifiers, and the like), flavoring agents, sweeteners, opacifying agents, buffering agents, tableting agents, tableting lubricants, preservatives, degredation enhancers, mucosal adhesive polymers, gastrorentive agents, and the like, and any combination thereof. An excipient may be a pharmaceutically acceptable excipient. As used herein, unless otherwise stated, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

Some non-limiting examples of additive(s) which may be used in a composition include sugars, such as, for example, lactose, glucose, sucrose, and the like; starches, such as, for example, corn starch, potato starch, and the like; cellulose, and its derivatives, such as, for example, sodium carboxymethyl cellulose, ethyl cellulose, cellulose acetate, and the like; powdered tragacanth; malt; gelatin; talc; excipients, such as, for example, cocoa butter, suppository waxes, and the like; oils, such as, for example, peanut oil, cottonseed oil, safflower oil, sesame oil, olive oil, corn oil, soybean oil, and the like; glycols, such as, for example, propylene glycol and the like; polyols, such as, for example, glycerin, sorbitol, mannitol, polyethylene glycol, and the like; esters, such as, for example, ethyl oleate, ethyl laurate, and the like; agar; buffering agents, such as, for example, magnesium hydroxide, aluminum hydroxide, and the like; alginic acid; pyrogen-free water; isotonic saline; Ringer's solution; ethyl alcohol; phosphate buffer solutions; and other non-toxic compatible substances employed in pharmaceutical formulations. (See, e.g., REMINGTON'S PHARM. SCI., 15th Ed. (Mack Publ. Co., Easton (1975)).

In various examples, a composition is a food composition, a nutraceutical composition, a pharmaceutical composition, or the like. In various examples, a composition is in the form of or is in the form of a pill, a capsule (which may be soft-filled capsule or hard-filled capsule), a tablet, dragees, bead(s), granule(s), or the like. A tablet may be a scored tablet. In various examples, a composition comprises nanoparticles in a solid form (e.g., a powder, such as, for example, a lyophilized powder). A composition may comprise a liquid comprising the nanoparticles (e.g., an aqueous or suspension or an aqueous solution of the nanoparticles, an aqueous ethanol suspension or an aqueous ethanol solution of the nanoparticles, or the like). An aqueous suspension or aqueous solution may comprise one or more buffering agent(s), one or more preservative(s), or the like, or a combination thereof. A composition may comprise a gel comprising the nanoparticles.

In an aspect, the present disclosure provides uses of the compositions. In various examples, compositions are used in oral delivery methods. In various examples, oral delivery methods are used to deliver one or more composition(s) for targeting, diagnosing, treating, preventing, or the like, or any combination thereof, of a current or future condition, disorder, disease, disease state, or the like, or any combination thereof, in an individual. In various examples, one or more composition(s) are used in oral delivery and/or imaging methods.

In various examples, a method of delivering a plurality of nanoparticles to an individual may comprise orally administering one or more composition(s) of the present disclosure to an individual. In various examples, at least a portion of, substantially all, or all of the nanoparticles are delivered to the post-stomach portion of the gastrointestinal tract (e.g., large intestine, small intestine, colon, ileum, or the like) of the individual.

In various examples, at least a portion of (e.g., 40% or more, 50% or more, 60% or more, or 70% or more), substantially all (e.g., at least 90%, at least 95%, or at least 99% of), or all of the nanoparticles orally delivered to the post-stomach portion of the gastrointestinal tract of an individual pass through the mucus layer and epithelial lining of the small intestine of the individual. In various examples, at least a portion of, substantially all, or all of the nanoparticles are in a form that retains at least a portion of, substantially all (e.g., at least 90%, at least 95%, or at least 99% of), or all of one or more or all of the activit(ies) of the nanoparticles upon oral delivery to the post-stomach portion of the gastrointestinal tract of an individual.

In various examples, a method of targeting, treating, diagnosing, or the like, or any combination thereof, a condition, disorder, disease, disease state or potential condition, disorder, disease or disease state, or the like, in an individual in need of treatment thereof comprises orally administering one or more composition(s) of the present disclosure to the individual (which may be an effective amount of the composition(s). In various examples, at least a portion of, substantially all, or all of the nanoparticles are delivered to the post-stomach portion of the gastrointestinal tract (e.g., large intestine, small intestine, colon, ileum, or the like) of the individual.

An individual (e.g., an individual in need of treatment or the like) may be a human or non-human mammal or other animal. Non-limiting examples of non-human mammals include cows, pigs, mice, rats, rabbits, cats, dogs, and other agricultural mammals, pets (such as, for example, dogs, cats, and the like), service animals, and the like.

“Treating” or “treatment” of any disease or disorder refers, in various examples, to ameliorating the disease, disease state, condition, disorder, side effect, or the like, or a combination thereof, (e.g., arresting, reversing, alleviating, or the like) the disease, disease state, condition, disorder, side effect, or the like, or a combination thereof, or reducing the manifestation, extent or severity of one or more clinical symptom(s) thereof, or the like). In various other examples, “treating” or “treatment” refers to ameliorating one or more physical parameter(s), which, independently, may or may not be discernible by the individual. In yet other examples, “treating” or “treatment” refers to modulating disease, disease state, condition, disorder, side effect, or the like, or a combination thereof, either physically, (e.g., stabilization of one or more discernible symptom(s), or the like), physiologically, (e.g., stabilization of one or more physical parameter(s), or the like), or both. In yet other examples, “treating” or “treatment” relates to slowing the progression of the disease, disease state, condition, disorder, side effect, or the like, or a combination thereof.

In various examples, a method of the present disclosure targets, diagnoses, treats, prevents, or the like, or any combination thereof, any current or potential condition, disease, disease state, or the like, or any combination thereof, that may be conventionally or traditionally targeted, diagnosed, treated, or prevented, or the like, or any combination thereof, with a targeting agent, therapeutic agent, diagnosing agent, or the like, or any combination thereof, that can be delivered using one or more composition(s) of the present disclosure. Non-limiting examples of diseases, disease states, conditions, disorders, side effects, and the like, and potential diseases, disease states, conditions, disorders, side effects, and the like, include infections (e.g., bacterial infections, viral infections, and the like), cancers, neurological conditions/diseases, neurodegenerative diseases, psychological conditions/diseases, inflammatory conditions/diseases, cardio-vascular diseases, and the like.

Typically, a composition is orally administered in an amount effective to target, diagnose treat, prevent, or the like, or any combination thereof, a current disease, disease state, condition, disorder, side effect, or the like, or a combination thereof, or a potential disease, disease state, condition, disorder, side effect, or the like, or a combination thereof, as described herein. As used herein, unless otherwise indicated, the term “effective amount” means that amount of a composition that will elicit the biological or medical response of a tissue, system, animal or human that is being sought, for instance, by a researcher, clinician, or the like.

A physician or veterinarian having ordinary skill in the art can readily determine and prescribe the effective amount of the composition required. Effective doses of the compounds required to target, diagnose, treat, prevent, or the like, or any combination thereof, the progress of the medical condition are readily ascertained by one of ordinary skill in the art using preclinical and clinical approaches familiar to the medicinal arts. In various examples, the selected dosage level depends upon a variety of factors including, but not limited to, the activity of the particular composition employed, the rate of excretion or metabolism of the particular composition being employed, the rate and extent of absorption, the duration of the treatment, other drugs, compounds and/or materials used in combination with the particular composition employed, the age, sex, weight, condition, general health and prior medical history of the patient being treated, and like factors well known in the medical arts. In various examples, the physician or veterinarian could start doses of the composition employed at levels lower than that required in order to achieve the desired therapeutic effect and gradually increase the dosage until the desired effect is achieved.

An effective amount may be a therapeutically effective amount. The term “therapeutically effective amount” includes any amount which, as compared to a corresponding subject who has not received such amount, results in improved treatment, healing, prevention, or amelioration of a disease, disease state, condition, disorder, side effect, or the like or a decrease in the rate of advancement of a disease, disease state, condition, disorder, or the like, or the like. The term also includes within its scope amounts effective to enhance normal physiological function. In various examples, the individual is considered effectively treated if the treated individual is not thereafter diagnosed with the disease or disease state, or one or more symptom(s), one or more indication(s), or the like of condition, disorder, disease, or disease state, or the like is at least partially or completely prevented, inhibited, alleviated, or the like).

An effective amount may result in prophylaxis. The term “prophylaxis” includes prevention and refers to a measure or procedure which is to prevent rather than cure or treat a disease. Preventing may refer to a reduction in risk of acquiring or developing a disease causing at least one clinical symptom of the disease not to develop in a subject that may be exposed to a disease-causing agent or a subject predisposed to the disease in advance of disease outset.

In various examples, imaging is used to determine the location of the nanoparticles in an individual. Following administration of one or more composition(s) the path, location, clearance of the nanoparticles, or the like, or a combination thereof, may be monitored using one or more imaging technique(s). Examples of suitable imaging techniques include, but are not limited to, Artemis Fluorescence Camera System and the like.

A method may comprise imaging of an individual. In various examples, a method comprises imaging at least a portion of (which may or may not be in the individual) or all of the individual. In various examples, a method for imaging of a region within an individual comprises, directing excitation light into the subject, thereby exciting at least one of one or more dye molecule(s); detecting excited light, the detected light having been emitted by said dye molecules in the individuals as a result of excitation by the excitation light; and (d) processing signals corresponding to the detected light to provide one or more image(s) (e.g. a real-time video stream) of the region within the subject. In various examples, for directing e/m radiation at cells, extracellular materials, tissues, organs and the like within an individual or any portion of an individual's body that are not easily accessible, fiber optical instruments are used. In various examples, following administration of a composition comprising the present nanoparticles, the path, location, and clearance of the nanoparticles are monitored using one or more imaging technique(s) such as, for example, the Artemis Near Infrared (NIR) Fluorescence Camera System or the like.

In various examples, an imaging method (e.g., imaging an individual or a portion thereof) comprise imaging fluorescence correlation spectroscopy (Imaging FCS), a total internal reflection fluorescence (TIRF) microscopy-based variant of FCS, (which may be referred to as a “leaky” TIRFM method). In various examples, the angle of the incident radiation is above the total internal reflection fluorescence angle. Without intending to be bound by any particular theory, it is considered that slightly changing the incident angle of the excitation light (“leaky” TIRF) allows maximal focus on diffusing particles (e.g., and not on those immobilized on a glass surface).

In various examples, additionally or alternatively, radioisotopes are further attached to the ligand groups (e.g., tyrosine residue or chelator) of the ligand-functionalized particles or to the silica matrix of the PEGylated particles without specific ligand functionalization for positron emission tomography (PET) imaging. If the radioisotopes are chosen to be therapeutic, such as ²²⁵Ac or ¹⁷⁷Lu, this in turn would result in particles with additional radiotherapeutic properties.

A method may further comprise one or more additional (or other) therapeutic modalit(ies). Non-liming examples of therapeutic modalities include conventional/traditional drug therapies, surgical intervention (e.g., one or more surgical procedure(s) and the like), chemotherapy, radiation, and the like. A method may further comprise one or more additional (or other) diagnostic modalit(ies). Non-liming examples of diagnostic modalities include conventional/traditional diagnostic tests, methods, or the like. In various examples, the diagnostic modality is an imaging method (e.g., CT imaging, MRI, PET, x-ray imaging, or the like), or the like. In various examples, the additional modalit(ies) is/are carried out before, after, or in concert with a method of the present disclosure.

The following Statements describe various examples of methods, products and systems of the present disclosure and are not intended to be in any way limiting:
Statement 1. A method of oral delivery, the method comprising: orally administering one or more composition(s) to an individual, each composition comprising a plurality of silica nanoparticles and/or aluminosilicate nanoparticles, wherein each silica nanoparticle or aluminosilicate nanoparticle independently comprises a silica core or an aluminosilicate core, respectively, and a plurality of polyethylene glycol (PEG) groups disposed on at least a portion of a surface or surfaces of the silica nanoparticle or the aluminosilicate nanoparticle, respectively, wherein the silica nanoparticles and/or the aluminosilicate nanoparticles have a longest linear dimension of about 20 nm or less, including all 0.1 nm values and ranges therebetween, and wherein at least a portion of the silica nanoparticles and/or the aluminosilicate nanoparticles are delivered to the post-stomach portion of the gastrointestinal tract of the individual.
Statement 2. The method of Statement 1, wherein the silica nanoparticles and/or the aluminosilicate nanoparticles have a longest linear dimension of about 7 nm or less, including all 0.1 nm values and ranges therebetween.
Statement 3. The method of Statement 1 or Statement 2, wherein at least a portion of the silica nanoparticles and/or the aluminosilicate nanoparticles pass through the mucus layer and permeate the epithelial lining of the post-stomach portion of the gastrointestinal tract of the individual.
Statement 4. The method of any one of Statements 1-3, wherein the silica nanoparticles and/or the aluminosilicate nanoparticles exhibit one or more or all of the following:
   a diffusion coefficient through the mucus layer (D_{mucus}) of from about 5 μm²/s to about 35 μm²/s , including all 0.1 μm²/s values and ranges therebetween;
   a diffusion coefficient through water (D_{water}) of from about 20 μm²/s to about 70 μm²/s , including all 0.1 μm²/s values and ranges therebetween; or
   a ratio of the diffusion coefficients through the mucus layer and water (D_{mucus}/D_{water}) of from about 0.1 to about 0.5, including all 0.01 diffusion coefficient ratio values and ranges therebetween.
Statement 5. A method according to any one of Statements 1-4, wherein at least a portion of the silica nanoparticles and/or the aluminosilicate nanoparticles comprise one or more targeting group(s), one or more diagnostic group(s), one or more therapeutic group(s), or any combination thereof, disposed on at least a portion of a surface or surfaces of the silica nanoparticles and/or the aluminosilicate nanoparticles and/or within at least a portion of the silica matrix or the aluminosilicate matrix of individual silica nanoparticles or aluminosilicate nanoparticles, respectively.
Statement 6. A method according to any one of Statements 1-5, wherein at least a portion of the silica nanoparticles and/or the aluminosilicate nanoparticles delivered to the post-stomach portion of the gastrointestinal tract of the individual retain at least a portion of one or more targeting activit(ies), one or more diagnostic activit(ies), one or more therapeutic activit(ies), or any combination thereof, of the silica nanoparticles and/or the aluminosilicate nanoparticles.
Statement 7. A method according to any one of Statements 1-6, wherein the composition(s) further comprise(s) one or more material(s) that render the composition(s) suitable for delivery of at least a portion of the silica nanoparticles and/or the aluminosilicate nanoparticles to the post-stomach portion of the gastrointestinal tract of the individual.
Statement 8. A method according to Statement 7, wherein the plurality of silica nanoparticles and/or aluminosilicate nanoparticles is disposed within the material(s).
Statement 9. A method according to Statement 7 or Statement 8, wherein at least a portion of the material(s) comprise(s) one or more enteric material(s).
Statement 10. A method according to any one of Statements 1-9, wherein the individual is an individual in need of targeting, diagnosing, treating, preventing, or any combination thereof, a current or potential disease, disease state, condition, disorder, side effect, or any combination thereof, and wherein the current or potential disease, disease state, condition, disorder, side effect, or any combination thereof, is targeted, diagnosed, treated, prevented, or any combination thereof, in the individual.
Statement 11. A method of targeting, diagnosing, treating, preventing, or any combination thereof, a current or potential disease, disease state, condition, disorder, side effect, or any combination thereof, in an individual, the method comprising: orally administering one or more composition(s) to the individual, the composition(s) comprising a plurality of silica nanoparticles and/or aluminosilicate nanoparticles, wherein each silica nanoparticle or aluminosilicate nanoparticle independently comprises a silica core or an aluminosilicate core, respectively, and a plurality of polyethylene glycol (PEG) groups disposed on at least a portion of a surface or surfaces of the silica nanoparticle or the aluminosilicate nanoparticle, respectively, wherein the silica nanoparticles and/or the aluminosilicate nanoparticles have a longest linear dimension of about 20 nm or less, including all 0.1 nm values and ranges therebetween, wherein at least a portion of the silica nanoparticles and/or the aluminosilicate nanoparticles are delivered to the post-stomach portion of the gastrointestinal tract of the individual and the current or potential disease, disease state, condition, disorder, side effect, or any combination thereof, is targeted, diagnosed, treated, prevented, or any combination thereof.
Statement 12. A method according to Statement 11, wherein the silica nanoparticles and/or the aluminosilicate nanoparticles have a longest linear dimension of about 7 nm or less, including all 0.1 nm values and ranges therebetween.
Statement 13. A method according to Statement 11 or Statement 12, wherein the current or potential disease, disease state, condition, disorder, side effect, or any combination thereof, is chosen from infections, cancers, neurological conditions/diseases, neurodegenerative diseases, psychological conditions/diseases, inflammatory conditions/diseases, cardio-vascular diseases, and any combination thereof.
Statement 14. A method according to any one of Statements 11-13, wherein at least a portion of the silica nanoparticles and/or the aluminosilicate nanoparticles comprise one or more targeting group(s), one or more diagnostic group(s), one or more therapeutic group(s), or any combination thereof, disposed on at least a portion of a surface or surfaces of the silica nanoparticles and/or the aluminosilicate nanoparticles and/or within at least a portion of the silica matrix or the aluminosilicate matrix of individual silica nanoparticles or aluminosilicate nanoparticles, respectively.
Statement 15. A method according to any one of Statements 11-14, wherein at least a portion of the silica nanoparticles and/or the aluminosilicate nanoparticles delivered to the post-stomach portion of the gastrointestinal tract of the individual retain at least a portion of one or more targeting activit(ies), one or more diagnostic activit(ies), one or more therapeutic activit(ies), or any combination thereof, of the silica nanoparticles and/or the aluminosilicate nanoparticles.
Statement 16. A method according to any one of Statements 11-15, wherein the individual is an individual in need of targeting, diagnosing, treating, preventing, or any combination thereof, of the current or potential disease, disease state, condition, disorder, side effect, or any combination thereof, and wherein the current or potential disease, disease state, condition, disorder, side effect, or any combination thereof, is targeted, diagnosed, treated, prevented, or any combination thereof, in the individual.
Statement 17. A method according to Statement 14-16, wherein the diagnostic group(s) is/are independently at each occurrence chosen from dye group(s), sensor group(s), radioisotope(s), and any combination thereof, and wherein the method further comprises obtaining one or more image(s) of the individual.
Statement 18. A method according to any one of Statements 11-17, wherein the method further comprising one or more additional targeting modalit(ies), one or more additional therapeutic modalit(ies), one or more additional diagnostic modalit(ies), or any combination thereof.
Statement 19. A method according to any one of Statements 11-18, wherein the composition(s) further comprise(s) one or more material(s) that render the composition(s) suitable for delivery of the silica nanoparticles and/or the aluminosilicate nanoparticles to the post-stomach portion of the gastrointestinal tract of the individual.
Statement 20. A method according to Statement 19, wherein the plurality of silica nanoparticles and/or aluminosilicate nanoparticles is disposed within the material(s).
Statement 21. A method according to Statement 19 or Statement 20, wherein at least a portion of the material(s) comprise(s) one or more enteric material(s).
Statement 22. An oral delivery composition comprising: a plurality of silica nanoparticles and/or aluminosilicate nanoparticles, wherein each silica nanoparticle or aluminosilicate nanoparticle independently comprises a silica core or an aluminosilicate core, respectively, and a plurality of polyethylene glycol (PEG) groups disposed on at least a portion of a surface or surfaces of the silica nanoparticle or the aluminosilicate nanoparticle, respectively, wherein the silica nanoparticles and/or the aluminosilicate nanoparticles have a longest linear dimension of about 20 nm or less, including all 0.1 nm values and ranges therebetween; and one or more material(s) that render(s) the composition suitable for delivery of at least a portion of the silica nanoparticles and/or the aluminosilicate nanoparticles to the post-stomach portion of the gastrointestinal tract of an individual to whom the composition has been orally administered.
Statement 23. wherein the silica nanoparticles and/or the aluminosilicate nanoparticles have a longest linear dimension of about 7 nm or less, including all 0.1 nm values and ranges therebetween.
Statement 24. An oral delivery composition according to Statement 22 or Statement 23, wherein the aluminosilicate nanoparticles comprise an Al/Si atomic ratio of from about 0.01% to about 30%.
Statement 25. An oral delivery composition according to any one of Statements 22-24, wherein at least a portion of the silica nanoparticles and/or the aluminosilicate nanoparticles comprise one or more targeting group(s), one or more diagnostic group(s), one or more therapeutic group(s), or any combination thereof, disposed on at least a portion of or all of a surface or surfaces of the silica nanoparticles and/or the aluminosilicate nanoparticles and/or within at least a portion of the silica matrix or the aluminosilicate matrix of individual silica nanoparticles or aluminosilicate nanoparticles, respectively.
Statement 26. An oral delivery composition according to Statement 25, wherein one or more of the PEG groups, one or more of the targeting group(s), one or more of the diagnosing group(s), one or more of the therapeutic group(s), or any combination thereof, is/are covalently bound via a linking group to one or more of the silica nanoparticles and/or the aluminosilicate nanoparticles.
Statement 27. An oral delivery composition according to Statement 25 or Statement 26, wherein one or more of the PEG groups comprise(s) one or more of the targeting group(s), one or more of the diagnosing group(s), one or more of the therapeutic group(s), or any combination thereof.
Statement 28. An oral delivery composition according to any one of Statements 25-27, wherein the targeting group(s) is/are independently at each occurrence chosen from antibody fragment(s), aptamer(s), protein(s)/peptide(s) c), nucleic acid(s), and any combination thereof.
Statement 29. An oral delivery composition according to any one of Statements 25-28, wherein the diagnostic group(s) is/are independently at each occurrence chosen from dye group(s), sensor group(s), radioisotope(s), and any combination thereof.
Statement 30. An oral delivery composition according to any one of Statements 25-29, wherein the therapeutic group(s) is/are independently at each occurrence chosen from drug(s), nucleic acid(s), biological material(s), radioisotope(s), and any combination thereof.
Statement 31. An oral delivery composition according to any one of Statements 22-30, wherein the composition further comprises one or more material(s) that render the composition(s) suitable for delivery of at least a portion of the silica nanoparticles and/or the aluminosilicate nanoparticles to the post-stomach portion of the gastrointestinal tract of the individual to whom the composition has been orally administered.
Statement 32. An oral delivery composition according to Statement 31, wherein at least a portion of the material(s) comprise(s) one or more enteric material(s).
Statement 33. An oral delivery composition according to Statement 31 or Statement 32, wherein at least a portion of the material(s) comprise(s) one or more polymeric material(s). Statement 34. An oral delivery composition according to Statement 33, wherein the at least a portion of the polymeric material(s) is/are one or more pH-sensitive polymeric material(s).
Statement 35. An oral delivery composition according to any one of Statements 31-34, wherein the material(s) coat the plurality of silica nanoparticles and/or aluminosilicate nanoparticles.
Statement 36. An oral delivery composition according to any one of Statements 22-35, wherein the composition further comprises one or more additional material(s) that render the composition suitable for sustained silica nanoparticle and/or aluminosilicate nanoparticle release, delayed silica nanoparticle and/or aluminosilicate nanoparticle release, controlled silica nanoparticle and/or aluminosilicate nanoparticle release, time-dependent silica nanoparticle and/or aluminosilicate nanoparticle delivery, or any combination thereof.
Statement 37. An oral delivery composition according to any one of Statements 22-36, wherein the composition further comprises one or more one or more pharmaceutically acceptable excipient(s).
Statement 38. An oral delivery composition according to any of Statements 22-37, wherein the composition is in the form of a pill, a capsule, a tablet, a dragée, a bead, or a granule.
Statement 39. A composition (e.g., an oral delivery composition), the composition comprising:
   a plurality of silica nanoparticles and/or aluminosilicate nanoparticles, each nanoparticle independently having a silica core or aluminosilicate core (e.g., an aluminosilicate core having an Al/Si atomic ratio of 0.01 to 30%, including all 0.01 at. % values and ranges therebetween) having a longest linear dimension (which may be a diameter or the like) of 20 nm or less (e.g., less than 20 nm, less than 15 nm, or less than 10 nm, or less than 5 nm), and each nanoparticle comprising a plurality of polyethylene glycol (PEG) groups disposed on (e.g., covalently bound to) at least a portion of or all of a surface or a portion of or all of the surfaces of the nanoparticle; and
   one or more material(s) that render the composition suitable for delivery of the silica/aluminosilicate nanoparticles to the post-stomach portion of the gastrointestinal tract (e.g., large intestine, small intestine, colon, ileum, or the like, or a combination thereof) of an individual to whom the composition has been orally administered (e.g., enteric material and the like),
wherein the plurality of silica/aluminosilicate nanoparticles is disposed within (e.g., are sequestered by, coated by, or the like) the one or more material(s).
Statement 40. A composition according to Statement 39, wherein at least a portion of or all of the silica/aluminosilicate nanoparticles comprise one or more targeting group(s), one or more therapeutic group(s), one or more diagnostic group(s), or the like, or a combination thereof, disposed on (e.g., covalently bound to) at least a portion of or all of a surface or a portion of or all of the surfaces of the individual silica/aluminosilicate nanoparticle(s).
Statement 41. A composition according to Statement 39 or Statement 40, wherein at least a portion of or all of the material(s) are one or more additional material(s).
Statement 42. A composition according to any one of Statements 39-41, wherein the composition further comprises one or more additive(s).
Statement 43. A composition according to any of Statements 39-42, wherein the composition is in the form of or is in the form of a pill, a capsule (which may be soft-filled capsule or hard-filled capsule), a tablet, dragees, bead(s), granule(s), or the like.
Statement 44. A method of delivering a plurality of nanoparticles to an individual comprising
orally administering one or more composition(s) of the present disclosure (e.g., one or more composition(s) of any of Statements 39-43) to the individual, wherein at least a portion of, substantially all, or all of the nanoparticles are delivered to the post-stomach portion of the gastrointestinal tract (e.g., large intestine, small intestine, colon, ileum, or the like) of the individual.
Statement 45. A method according to Statement 44, wherein at least a portion of (e.g., 40% or more, 50% or more, 60% or more, or 70% or more) or all of the nanoparticles pass through the mucus and epithelial lining of the small intestine of the individual.
Statement 46. A method according to Statement 44 or 45, wherein at least a portion of, substantially all, or all of these nanoparticles are in a form that retains at least a portion of, substantially all (e.g., at least 90%, at least 95%, or at least 99% of), or all of one or more or all of the activit(ies) (e.g., diagnostic activit(ies), therapeutic activit(ies), or the like) of the nanoparticles.
Statement 47. A method of treating an individual in need of treatment for a disease, disease state, condition, disorder, side effect, or the like or disease, disease state, condition, disorder, side effect, or the like, or the like, the method comprising
orally administering one or more composition(s) of the present disclosure (e.g., one or more composition(s) of any of Statements 39-43) to the individual (which may be an effective amount of the composition(s)),
wherein at least a portion of, substantially all, or all of the nanoparticles are delivered to the post-stomach portion of the gastrointestinal tract (e.g., large intestine, small intestine, colon, ileum, or the like) of the individual.
Statement 48. A method according to Statement 47, wherein at least a portion of (e.g., 40% or more, 50% or more, 60% or more, or 70% or more) or all of the nanoparticles pass through the mucus and epithelial lining of the small intestine of the individual.
Statement 49. A method according to Statement 47 or 48, wherein at least a portion of, substantially all, or all of these nanoparticles are in a form that retains at least a portion of, substantially all (e.g., at least 90%, at least 95%, or at least 99% of), or all of one or more or all of the activit(ies) (e.g., diagnostic activit(ies), therapeutic activit(ies), or the like) of the nanoparticles.
Statement 50. A method according to one of Statements 47-49, wherein at least a portion of the nanoparticles comprises one or more therapeutic group(s).
Statement 51. A method according to one of Statements 47-50, wherein one or more of the nanoparticle(s) comprises one or more dye group(s), one or more radioisotope(s), or the like, or a combination thereof, and the method further comprises obtaining one or more image(s) of the individual.
Statement 52. A method according to any one of Statements 47-51, the method further comprising one or more additional therapeutic modalities.
Statement 53. A method according to any one of Statements 47-52, the method further comprising one or more additional diagnostic modalities.

The steps of the methods described in the various embodiments and examples disclosed herein are sufficient carry out the methods of the present disclosure. Thus, in an embodiment, a method consists essentially of a combination of the steps of the methods disclosed herein. In another embodiment, a method consists of such steps.

The following examples are presented to illustrate the present disclosure. They are not intended to be limiting in any matter.

### EXAMPLE

This example provides a description of compositions and methods the present disclosure.

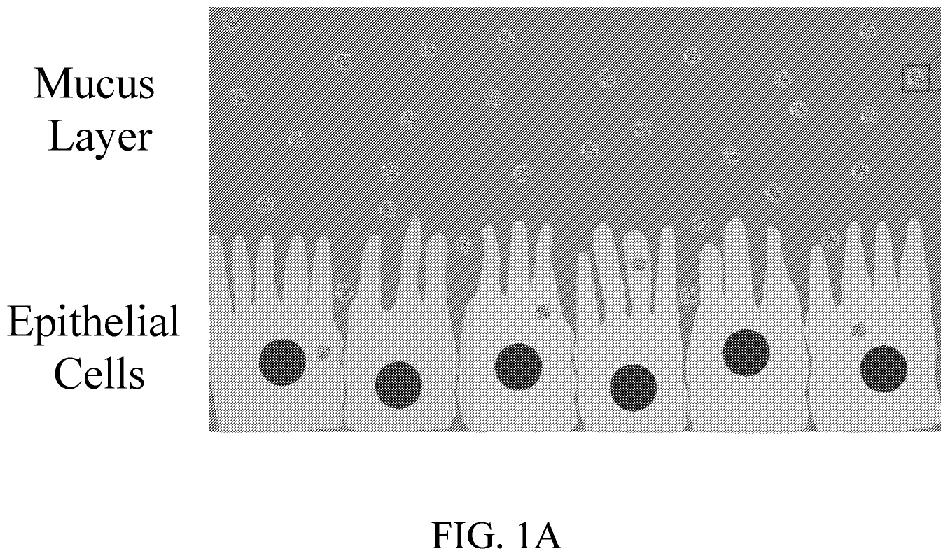

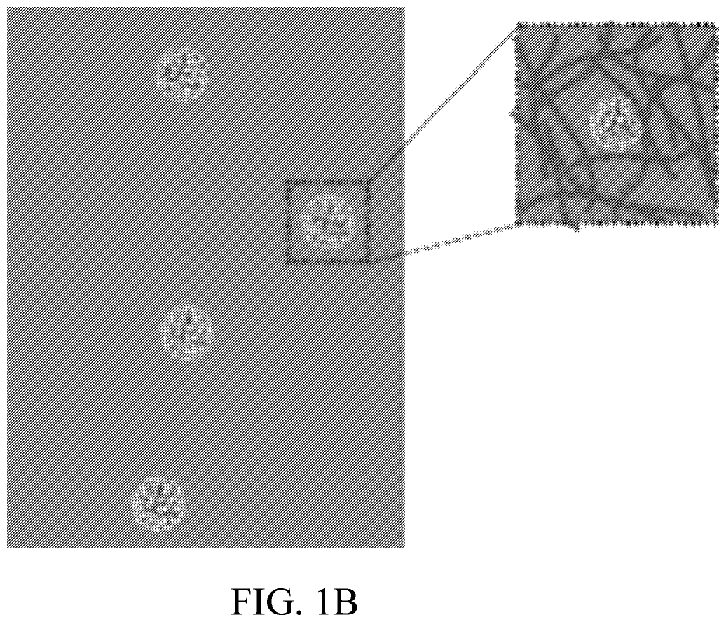

Nanoparticle-based drug delivery is a promising and safer alternative to intravenous injection. Unfortunately, the challenges involved in developing a successful oral formulation are difficult to overcome, in particular for nanoparticle formulations. It is well 30 accepted that the mucus in the small intestine as well as the epithelial lining of the small intestine itself are the critical barriers limiting oral delivery of nanomaterials (FIGS. 1A-1B).

How fluorescent PEGylated core-shell silica nanoparticles in the 5-50 nm size range interact with these two barriers was systematically studied. A mucus reconstitution protocol was first presented which is highly reproducible and accessible to non-experts. Next, image fluorescence correlation spectroscopy (Image FCS) on a quasi-(“leaky-”) TIRF microscope setup was established as a robust characterization method to determine the diffusivity of these fluorescent particles through the strongly scattering mucus environment. Two size regimes were found with a cutoff around 20 nm beyond which particle diffusion in mucus is progressively decreasing. Finally, the permeability of these different-sized nanoparticles was studied through Caco-2 cell monolayers mimicking the epithelial lining of the small intestine. It was found that ultrasmall core-shell silica nanoparticles (<10 nm) exhibit enhanced Caco-2 monolayer permeabilities above 1×10⁻⁶ cm/s likely attributed to both passive and active transport mechanisms. It was also observed that PEGylated ultrasmall silica nanoparticles exhibit high stability in artificial gastric juice. These findings, along with the formulation of a nanoparticle-loaded enterically coated pill to protect possible sensitive ligands from the acidic stomach environment, evidence that ultrasmall fluorescent core-shell silica nanoparticles, in particular Cornell Prime Dots (C′ dots) already in human clinical trials, have the potential for oral administration to patients.

Disclosed herein are ultrasmall and fluorescent silica core-poly(ethylene glycol) (PEG) shell (core-shell) nanoparticles synthesized in aqueous solutions termed Cornell Prime Dots (or C′dots) for biomedical applications. The C′ dot platform has been well characterized and shown to exhibit size control down to a single atomic silica layer through control of aqueous synthesis conditions, low particle size dispersity, and homogenous surface characteristics for diameters below 10 nm. Their ultrasmall size enables for intravenously injected C′ dots to avoid substantial RES uptake and be renally cleared. These particles are currently in multiple human clinical trials (e.g., see clinicaltrials.gov identifiers: NCT01266096, NCT02106598, and NCT04167969). Overall, their favorable biodistribution and pharmacokinetics characteristics make C′ dots a promising platform for next-generation drug delivery strategies.

In this Example, how fluorescent PEGylated core-shell silica nanoparticles with sizes ranging from ˜5-50 nm, i.e., including ultrasmall C′ dots, overcome the mucus and epithelial lining barriers that inhibit silica nanoparticle oral delivery was systematically studied. To that end, in the first part it was shown that imaging fluorescence correlation spectroscopy (Imaging FCS), a total internal reflection fluorescence (TIRF) microscopy-based variant of FCS, can be used with excellent reproducibility and fidelity to study fluorescent nanoparticle diffusion through strongly scattering mucus layers. The application of this technique decreases the sample preparation and analysis time substantially as compared to other methods of analysis such as SPT. In these experiments, a previously uncharacterized size-based diffusion cutoff in the mucus network was observed at ˜20 nm. In the second part, with three distinct particle sizes, one below, one similar to, and one significantly above the ˜20 nm cutoff observed in the mucus diffusion experiments, the size-dependent permeability across Caco-2 cell monolayers was studied. These investigations evidence that in particular ultrasmall C′dots have high enough permeability to be viable candidates for effective transport through the intestinal epithelial lining. In the third and last part, desirable stability of C′ dots in gastric juice was demonstrated and an enteric coating-based pill formulation for C′ dots was developed in order to protect possibly sensitive ligands susceptible to degradation by the acidic stomach environment. Results demonstrate that from all materials tested, ultrasmall PEGylated C′ dots exhibit the most optimal diffusion characteristics in mucus and the highest permeability across Caco-2 cells, and high stability in artificial gastric juice. With the development of an enterically coated pill-formulation, this work suggests that ultrasmall and fluorescent PEGylated core-shell silica nanoparticles (C′ dots) are a promising nanoparticle platform for oral administration to patients.

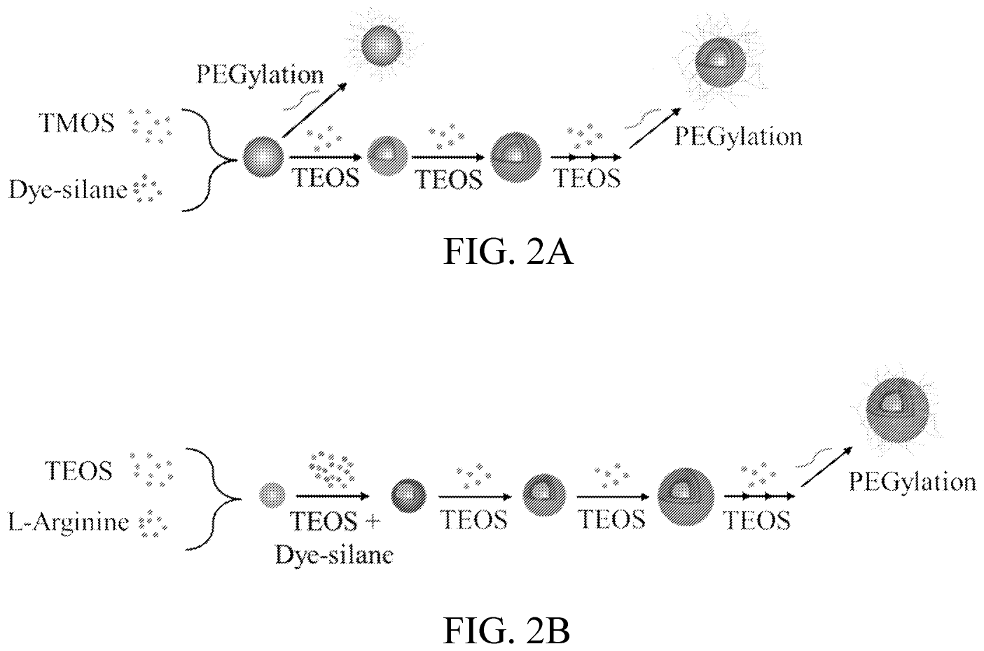

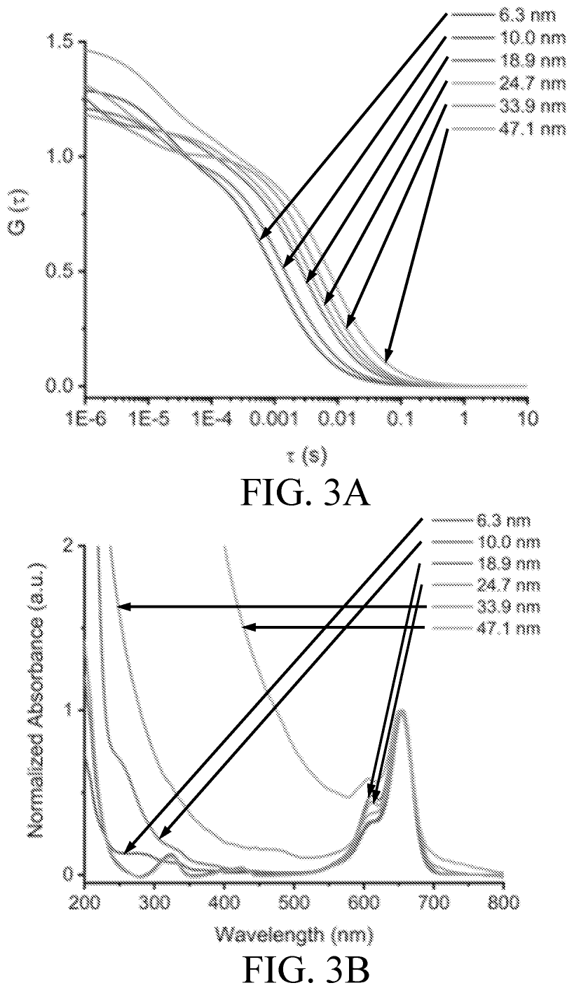

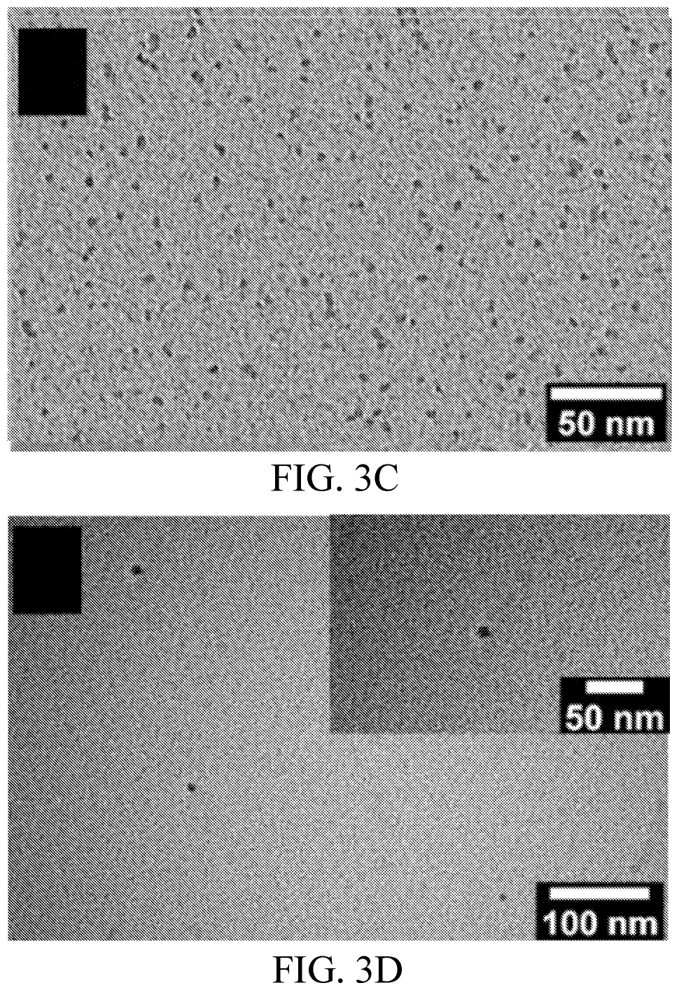

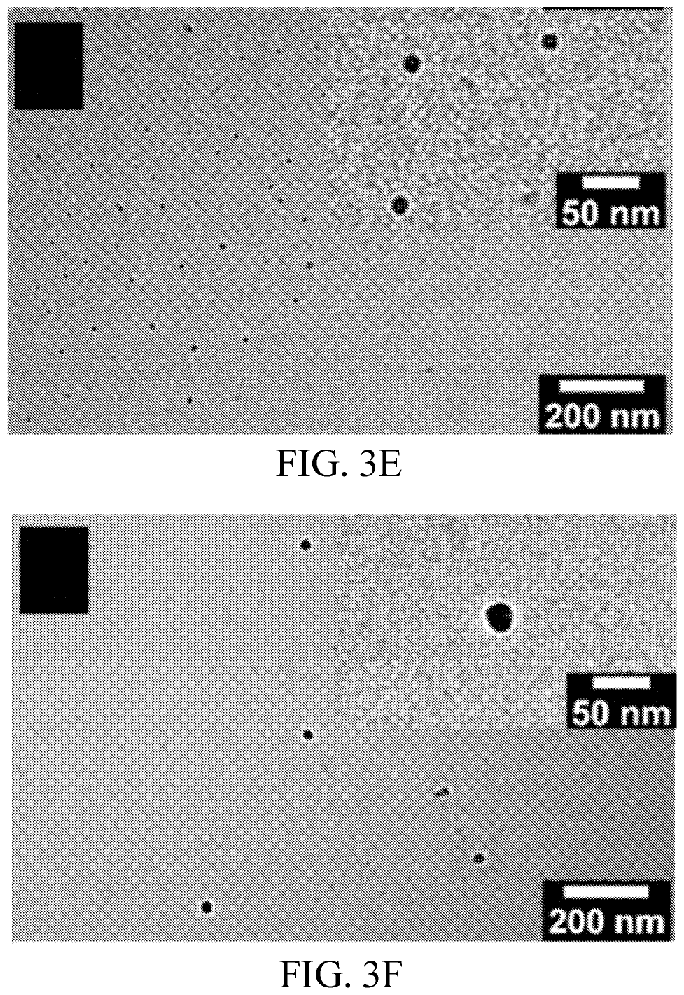

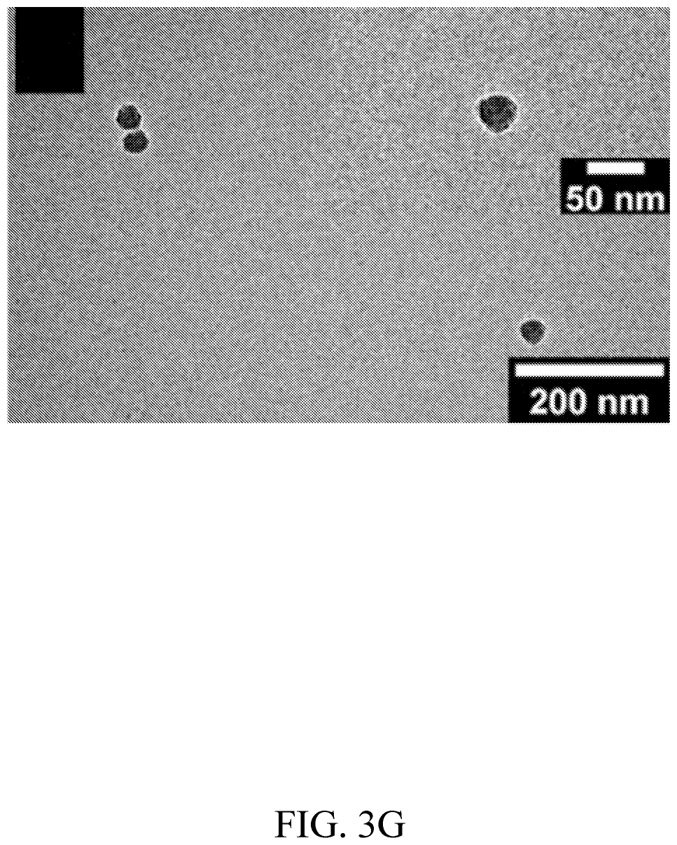

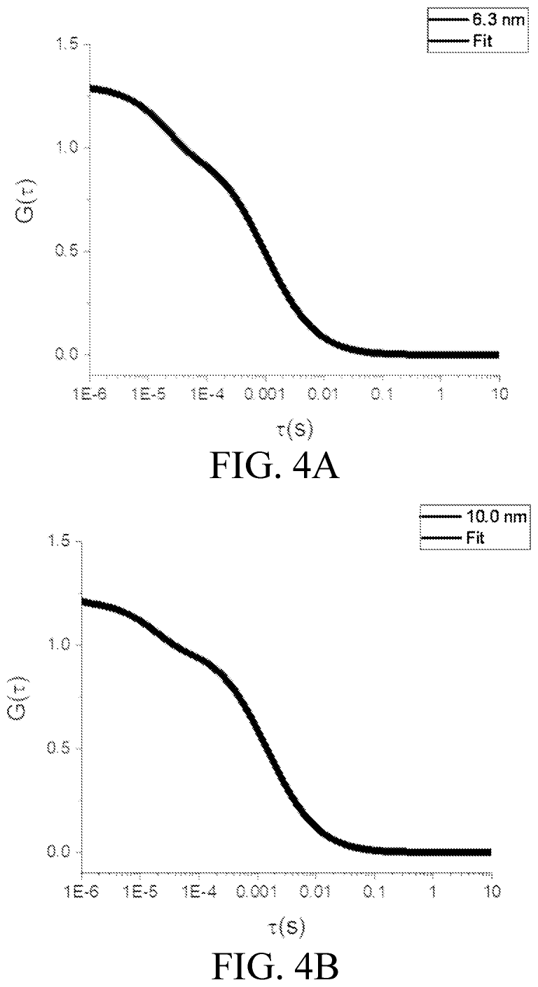

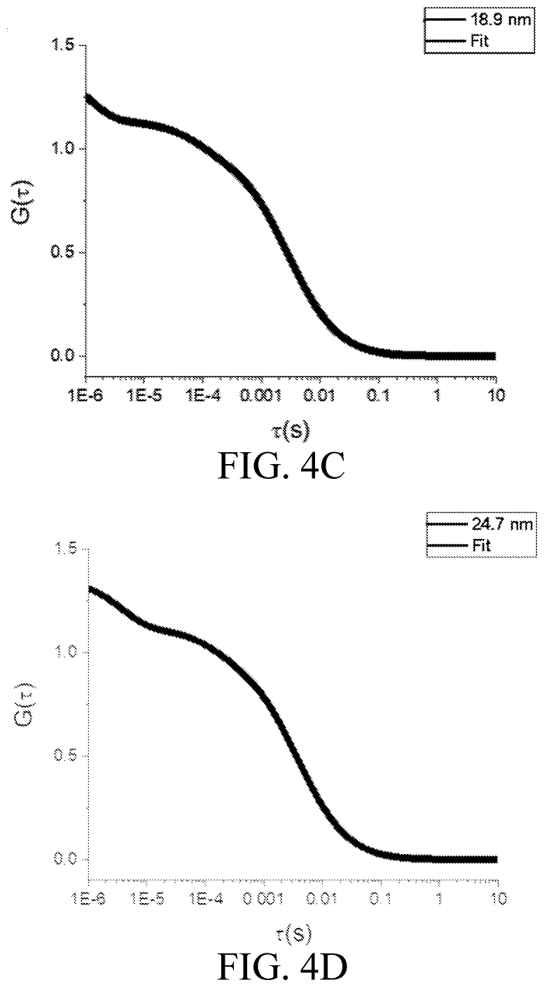

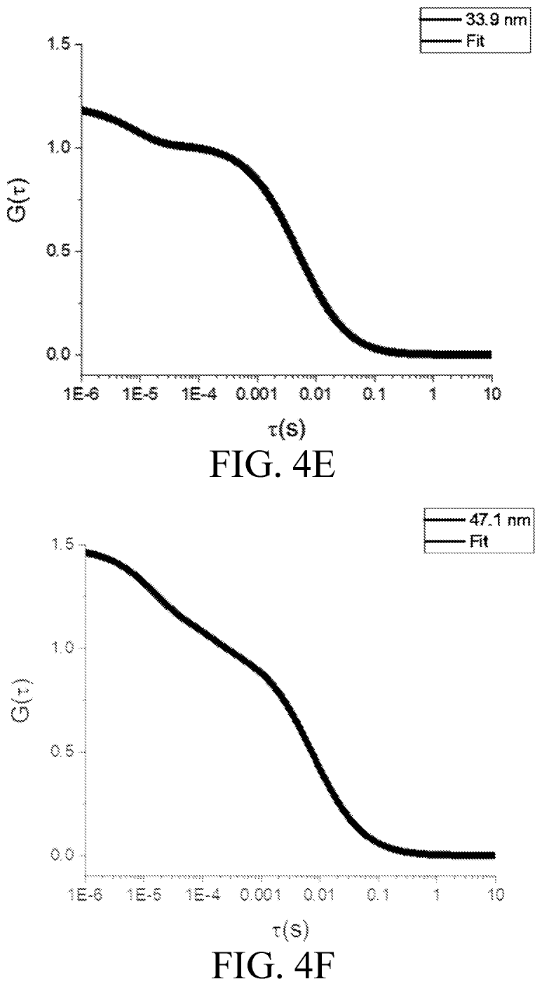

Nanoparticles of Varying Size: Synthesis and Characterization. Fluorescent PEGylated silica nanoparticles were synthesized via two main synthesis pathways as described in previous reports and illustrated in FIGS. 2A-2B. Smaller PEGylated silica nanoparticles with hydrodynamic diameters below 30 nm, as determined by confocal FCS (FIG. 3A), of 6.3 nm, 10.0 nm, 18.9 nm, and 24.7 nm (FIG. 2A) were synthesized with the same method originally developed to achieve ultrasmall C′ dots. In short, tetramethoxysilane as well as an organic dye-silane conjugate (Cy5-silane or ATTO647N-silane) were co-condensed in aqueous solution at a pH˜8. For the smallest set of nanoparticles of 6.3 nm, the growth was terminated by the addition of PEG-silane. For the 10.0 nm, 18.9 nm, and 24.7 nm sets of nanoparticles, silica shells were added to the un-PEGylated particle cores by dosing tetraethoxysilane (TEOS) until the desired size was reached, followed by PEGylation with an oligomeric PEG-silane with 6-9 ethylene oxide units (PEG(6-9)). Both sets of particles were then heated at 80° C. for 24 hours to aid in the PEGylation process. These particles were subsequently purified via gel permeation chromatography (GPC) and characterized with confocal FCS and UV/Vis spectroscopy (FIGS. 3A-3B) to obtain average hydrodynamic size, concentration, and dyes per nanoparticle as described in previous studies. Confocal FCS correlation curves for different sized particles with their respective fits are shown in FIG. 4. As expected, with increasing particle size, the FCS correlation curves in FIG. 3A move to longer time scales as it takes larger particles longer to diffuse through the same focal volume of the objective of the confocal FCS. At the same time, larger particles scatter more light, which is clearly observed by the increasing scattering background with size in the optical absorbance spectra in FIG. 3B. Finally, transmission electron microscopy (TEM, FIGS. 3C-3F) demonstrates the close to spherical shape of the different particles as well as their low size dispersity.

Larger PEGylated silica nanoparticles with hydrodynamic diameters between 30 nm and 50 nm, i.e. of 33.9 nm and 47.1 nm sizes, were synthesized with a seeded growth method using l-arginine as the base catalyst (FIG. 2B). Briefly, TEOS was carefully added in four sequential steps to the surface of an aqueous solution containing l-arginine at 60° ° C. Since TEOS does not mix with water, it forms a thin TEOS layer above the water solution, allowing for interfacial hydrolysis of TEOS only thereby controlling the rate of active silicic acid monomer formation entering the aqueous phase. Seed particle growth is further controlled by l-arginine, which does not only act as a catalyst, but also associates with the silica particle surface thereby lowering the condensation rate onto the particle increasing the degree of control over the process. After TEOS based seed formation, Cy5-silane was added dropwise to the seed solution followed by further TEOS addition to the aqueous solution surface in order to grow further silica shells and reach the desired particle diameter. Finally, PEG-silane was added and the solutions heated at 80° C. for 12 hours followed by the same purification and characterization steps as described above (FIG. 3G, FIGS. 4E-4F). TEM on the 47.1 nm nanoparticles is not available at this time. As is evident from FCS, UV/Vis, and TEM data sets in these figures, the even larger particles nicely follow the trends set by the smaller particles as a function of particle size.

An important aspect of these nanoparticles to note is the fact that they all have an oligomeric PEG shell. In addition to providing steric stability, as demonstrated in previous studies the neutral surface PEGylation allows for less hindered diffusion through mucus layers as a result of the lack of strong electrostatic interactions. Most past studies with silica nanoparticles for oral applications were with non-PEGylated particles, which may contribute to the limited number of silica nanoparticles that have successfully been administered orally.

Mucus Preparation. To begin the investigation into the diffusion of sub 50 nm nanoparticles in mucus, reconstituted porcine gastric mucin was first prepared according to previously published protocols and rheological results were compared to those in the literature. As previously shown, the viscoelastic properties of mucus are strongly pH dependent, so mucus adjusted to a neutral pH of around 7.4 was focused on to coincide with the physiologically relevant pH found in the majority of the small intestine. To ensure that the reconstituted mucus had similar properties to that of freshly harvested mucus, rheology was performed on five independently reconstituted and pH adjusted mucus samples and then the data was compared to existing literature data for freshly harvested mucus (Table 1).

TABLE 1

| | | Mucus | w | | G* | G′ | G″ |
|---|---|---|---|---|---|---|---|
| | Animal | Type | (rad/s) | η | (Pa) | (Pa) | (Pa) |
| Litera- | Pig | small | 0.01- | 0.063- | 0.19- | 0.18- | 0.05- |
| ture | | intestine | 100 | 5 | 12 | 10 | 6.3 |
| Experi- | Pig | recon- | 0.1- | 0.0831- | *** | 0.821- | 0.826- |
| mental | | stituted | 100 | 11.1 | | 5.05 | 6.44 |
| | | gastric | | | | | |
| | | mucin | | | | | |

*** Not available

Reasonably good agreement was observed between the two types of mucus and it was decided to continue with reconstituted porcine gastric mucins as the test mucus for diffusion studies. Previously, other studies concerning the diffusion of nanoparticles have used fresh undiluted mucus collected from healthy human volunteers. While this is the ideal medium in which to test the diffusion of nanoparticles, it is not always readily accessible. The low cost, rapid reconstitution, and the near unlimited supply of porcine gastric mucin make it attractive for initial nanoparticle screening before moving on to more complicated model systems.

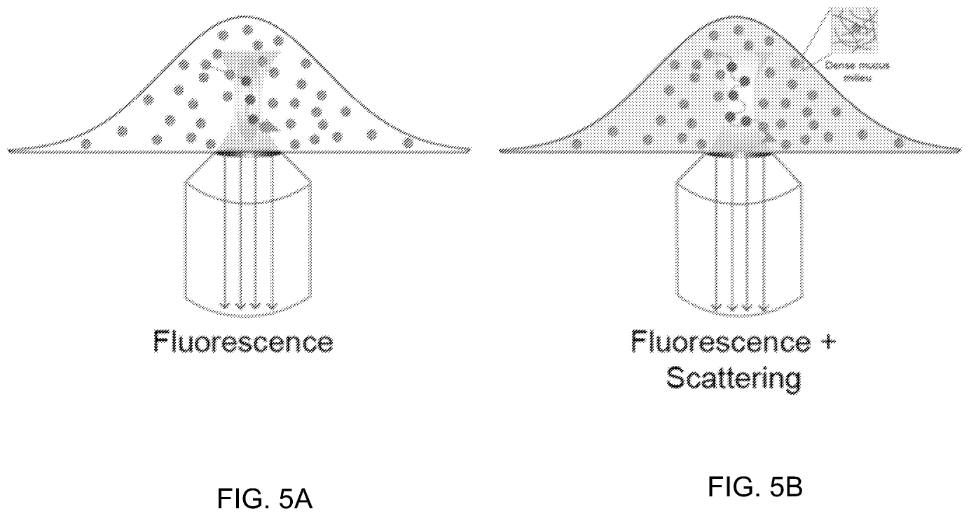

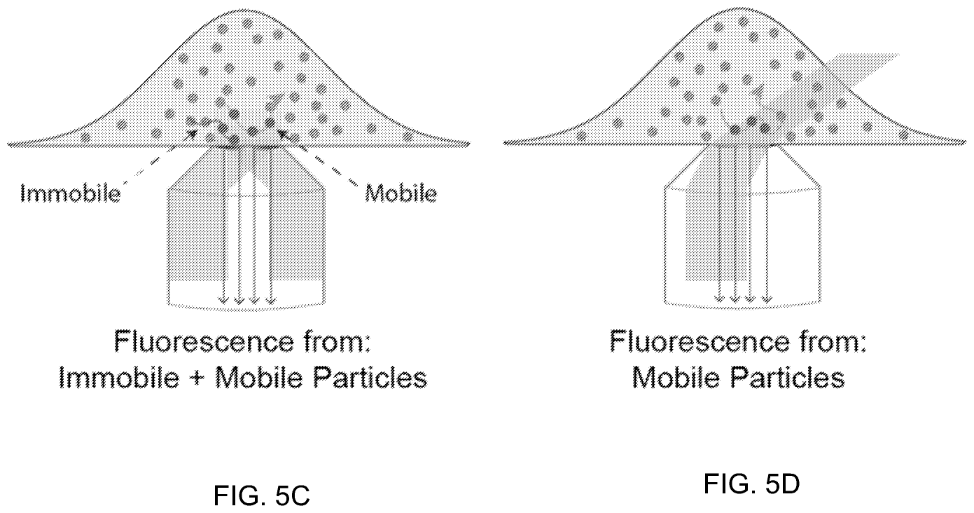

Nanoparticle Diffusion in Water and Mucus. When designing nanoparticles for oral delivery, their diffusivity in intestinal mucus is important as faster diffusion has been shown to lead to higher probability of reaching the epithelial lining before the mucus is cleared and replaced in the GI tract. To analyze the diffusion of nanoparticles in mucus, imaging FCS was employed which measures average diffusion of an ensemble of particles in multiple independent sample spots simultaneously. Such high throughput measurements greatly improve the statistics of diffusion data and thus the precision of diffusion property assessments. As was demonstrated in FIG. 3A and further illustrated in FIG. 5A, confocal FCS is routinely used to measure diffusion of fluorescent nanoparticles in water. Confocal FCS measurements in mucus are much more complex, however. Due to its dense and heterogeneous composition, mucus is a highly scattering environment, making confocal FCS nearly impossible due to the distortion of the ellipsoidal focal volume by the scattering medium and the high scattering background (FIG. 5B). To overcome this issue, it was decided to use imaging FCS, which rests on sample illumination by total internal reflection fluorescence (TIRF) microscopy and fluorescence detection by an electron multiplying charge-coupled device (EMCCD) camera. Unlike confocal FCS that involves performing autocorrelations of fluorescence signal time traces from a specified focal volume to obtain diffusion information, imaging FCS involves performing autocorrelations of pixel-based intensity information from a time series of several consecutive camera images recorded on a microscope. The time series can be analyzed pixel by pixel using readily available plugins in FIJI/ImageJ, and key parameters for the multiple fluorescent diffusing analytes can be determined simultaneously, such as the diffusion constant of the material. Furthermore, the optical sectioning provided by TIRF illumination was sufficient to detect nanoparticles above background noise arising from the highly scattering mucus medium.

Under TIRF microscopy illumination it was originally observed, however, that a significant subset of the silica nanoparticles adhered to the surface of the silicate glass substrate used in our imaging dish (FIG. 5C). Fluorescence from these immobile particles contaminate the fluorescence fluctuations of the diffusing particles making FCS analysis difficult. To limit this adhesion, the surface of the imaging dish was coated with a dense PEG layer, but this did not completely eliminate the problem. In order to be able to still take advantage of the beneficial signal-to-noise ratio from TIRF, but at the same time to maximize signal contributions from nanoparticles above the plane of the glass substrate, a quasi-TIRF imaging FCS setup was employed (FIG. 5D). By slightly changing the incident angle of the excitation light (“leaky” TIRF), it was possible to maximally focus on diffusing particles and not on those immobilized on the glass surface. At the same time, the axial depth of this setup was still limited allowing for us to decrease the impact of the high scattering background of the mucus samples.

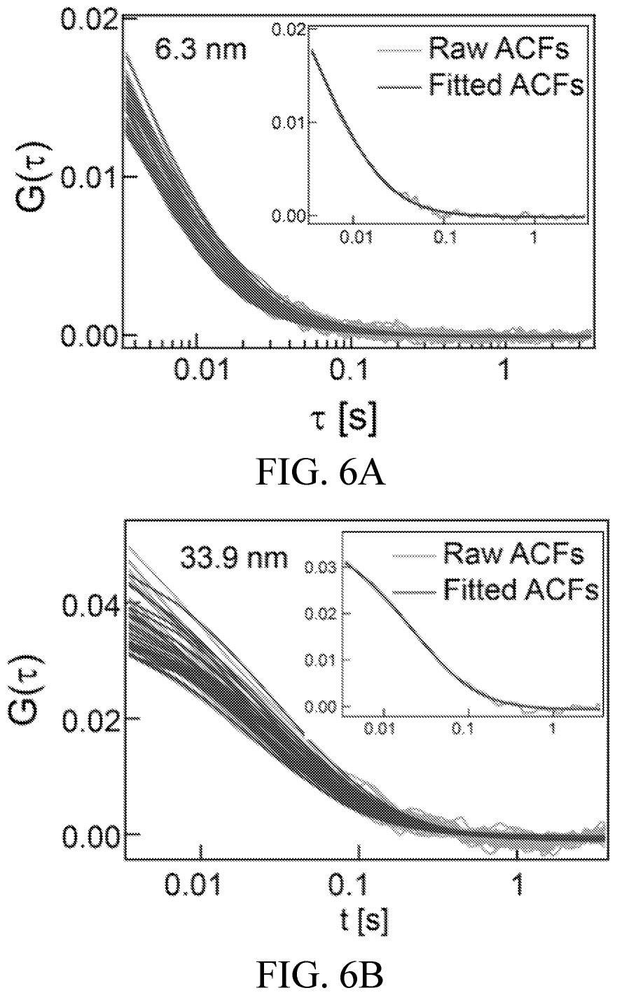

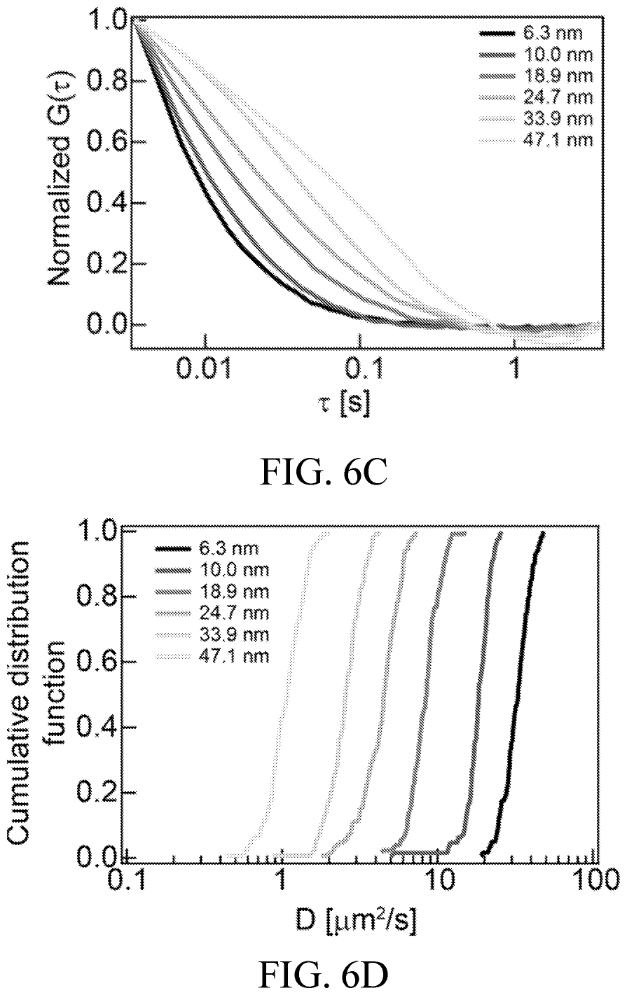

With this leaky TIRF setup, a stack of 20000 frames were recorded with an integration time of 3.5 ms for the differently sized nanoparticles diffusing in reconstituted mucus. Since the major contribution to fluorescence fluctuations in this setup comes from diffusion, performing autocorrelations of the fluorescence fluctuations of the entire stack as a function of time allowed calculation of the diffusion constant (D_{mucus}) by fitting to diffusion models described herein. As is apparent from comparison of results for two specific particle sizes of 6.3 nm and 33.9 nm, (FIGS. 6A-6B), as well as from the average autocorrelation functions for all nanoparticle sizes measured (FIG. 6C), there is a clear increase in lag time, τ, associated with increasing particle diameter: Larger particles diffuse slower in mucus than smaller ones, as expected. As the cumulative distribution functions of the resulting values of D_{mucus} from each individual measurement for each of the differently sized nanoparticle batches demonstrates (FIG. 6D), the average values of D_{mucus} for the different nanoparticle sizes are separable and quantifiable.

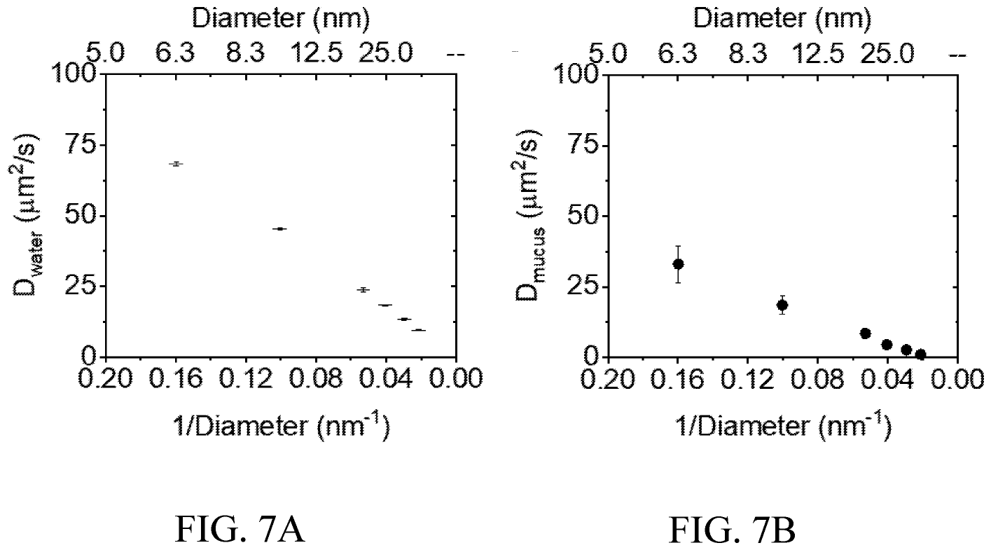

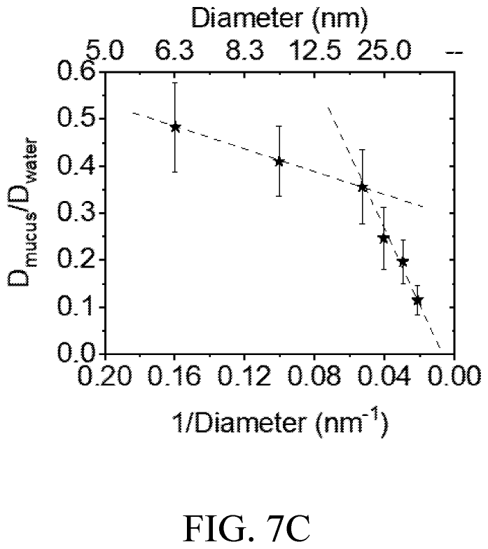

When plotting the diffusion coefficients of the nanoparticles in water, D_{water}, as obtained from fits of confocal FCS derived autocorrelation functions shown in FIG. 6A, as a function of inverse diameter, as expected based on the Stokes-Einstein Equation, the data exhibited a linear relationship (FIG. 7A). Furthermore, as expected due to the higher viscosity of mucus than water, the experimental values of the diffusion coefficients of the nanoparticles in mucus as derived from imaging FCS, D_{mucus}, for each specific particle size, were below that of D_{water} (compare FIGS. 7A-7B). Similar to the nanoparticles diffusing in water, in this second plot as a function of 1/diameter, D_{mucus} also monotonically decreased with increasing nanoparticle size. However, when the ratio of D_{mucus}/D_{water} (FIG. 7C) was taken, which signifies how fast a nanoparticle diffuses in mucus relative to water, two distinct regimes of particle behavior with two different apparent slopes were observed. There appeared to be a cutoff nanoparticle diameter of ˜20 nm. Nanoparticles with smaller diameters followed a linear trend for D_{mucus}/D_{water} decreasing more slowly as nanoparticle diameter increased when compared to nanoparticles with diameters greater than ˜20 nm. This suggests that nanoparticles with diameters<20 nm exhibit significantly improved diffusion characteristics in mucus when compared to nanoparticles with larger sizes. Furthermore, the values of D_{mucus} of ˜33 um²/s and associated ratio, D_{mucus}/D_{water}, of ˜0.4 for the smallest nanoparticle measured of 6.3 nm hydrodynamic diameter are high enough to indicate that these particles can diffuse through the mucus layer faster than it is replaced.

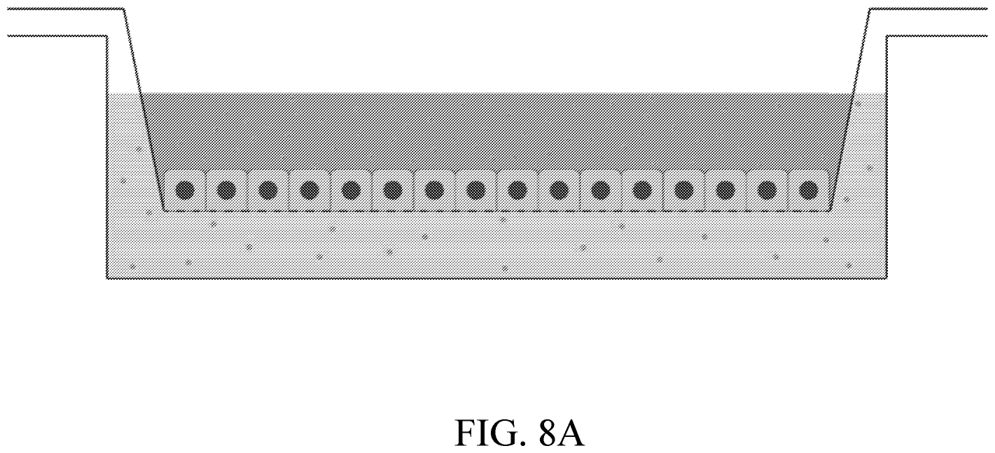

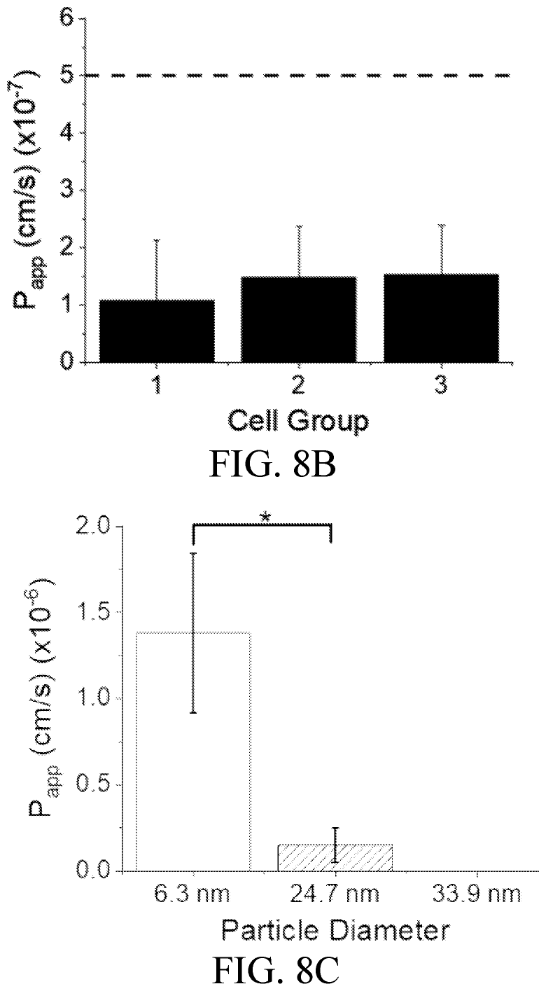

Nanoparticle Permeability Through Caco-2 Monolayers. As mentioned earlier, compounds that exhibit increasing permeability through a Caco-2 monolayer have been shown to correlate with higher uptake into the bloodstream when administered orally in human patients. The standard assay used to study the permeability of compounds through a Caco-2 monolayer involves growing the cells in a Transwell dish on a semipermeable membrane (FIG. 8A). After the cells have formed a monolayer, the compound of interest is added to the apical side (on top of the cells) at a known concentration and allowed to incubate with the cells for a specified amount of time. Following the incubation, the concentration of the compound of interest in the basolateral side is measured. Then equation (1), where dQ/dt is the molar flux into the basolateral side, A is the surface area of the semi-permeable membrane, and C₀ is the initial concentration on the apical side, is used to determine the apparent permeability (Pₐₚₚ) of that compound across the Caco-2 monolayer, with higher Pₐₚₚ values indicating higher permeability, and therefore higher probability of uptake across the intestinal lining.

$$\\ \begin{matrix}{P}_{app} = \frac{dQ}{dt} \times \frac{1}{A \times {C}_{0}} & ( 1 )\end{matrix}$$

To test that the monolayer well resembled the epithelial lining of the GI tract and formed tight junctions, Lucifer Yellow (LY) was used as a standard. It has been reported that the integrity of a Caco-2 monolayer is good once the Pₐₚₚ of LY across the monolayer is <5×10⁻⁷ cm/s. The measured Pₐₚₚ of LY across all Caco-2 monolayers (see Methods for more details) used in this study is depicted in FIG. 8B, with all values of Pₐₚₚ<3×10⁻⁷ cm/s.

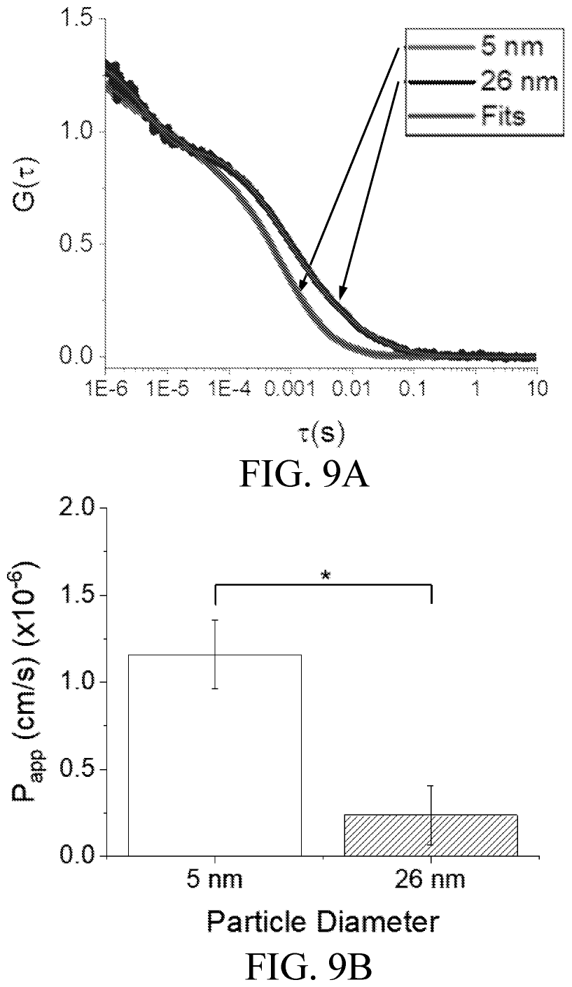

First the apparent permeability of the nanoparticles with 6.3 nm, 24.7 nm, and 33.9 nm hydrodynamic diameter used in the diffusion studies was examined. In FIG. 8C, resulting values for Pₐₚₚ of the nanoparticles across the Caco-2 monolayer significantly decrease as particle size increases. This study was replicated with nanoparticles of similar sizes (i.e. 5 nm and 26 nm) on a different Caco-2 passage, and very similar results were observed (FIGS. 9A-9B). This relationship has been studied previously, but not with particles of this ultrasmall size. The Pₐₚₚ observed for the ultrasmall C′ dots is similar to that of other ultrasmall nanoparticles with different compositions. The finding that silica nanoparticles, particularly the ultrasmall ones with diameters<10 nm, can diffuse across Caco-2 monolayers with permeabilities above 1×10-6 cm/s is significant as no other silica nanoparticle is believed to have been demonstrated so far to be able to permeate across these cells. As mentioned earlier, the silica nanoparticles that have been studied to date were >20 nm in diameter and were not PEGylated, both likely contributing factors to their inability to traverse these monolayers. Finally, it was observed that nanoparticles with a diameter well above 25 nm had a Pₐₚₚ below the detection limit of this cell system. It is interesting to note that this roughly agrees with the size threshold around 20 nm observed for the diffusion of nanoparticles through reconstituted mucus (FIG. 7C).

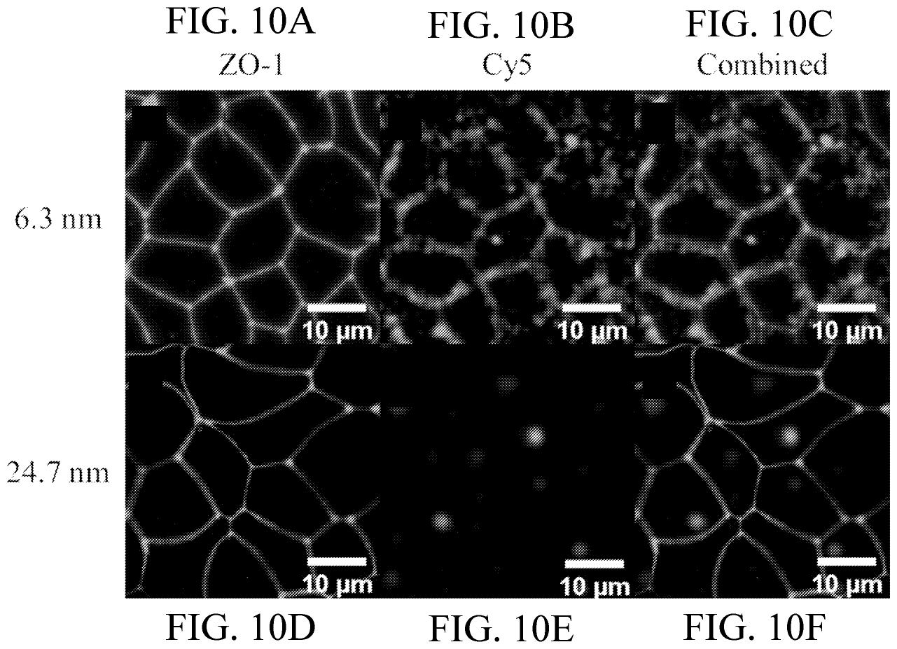

To better understand the mechanism by which these particles traverse the Caco-2 cell monolayer, the Caco-2 permeability experiments were repeated using the 6.3 nm and 24.7 nm particles, fixed the cells and immunostained the ZO-1 tight junction protein, and performed fluorescence laser scanning confocal microscopy to visualize the localization of these particles in relation to the tight junctions. From FIG. 10A-10C, the ultrasmall C′ dots

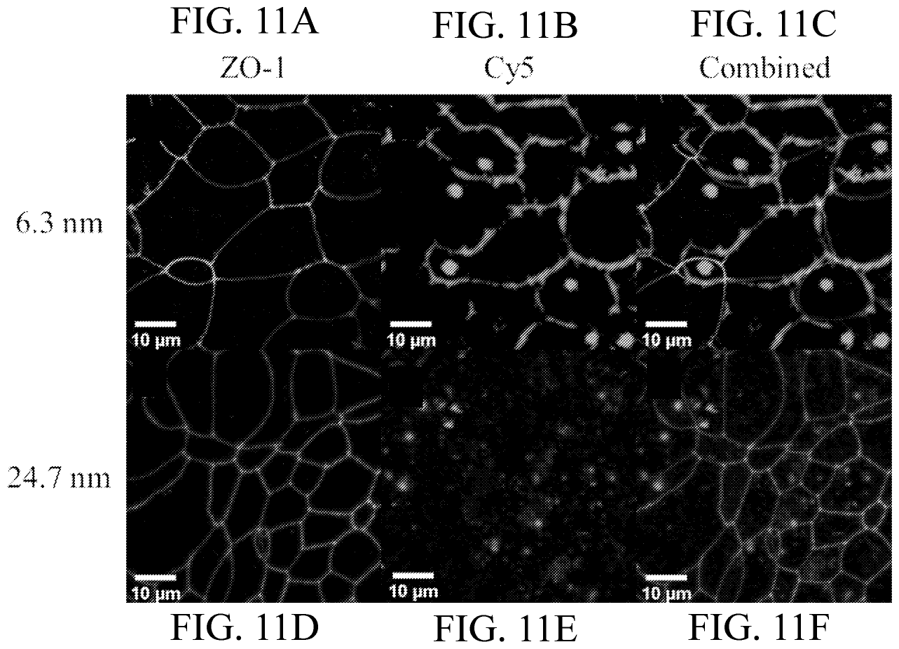

(Cy5) preferentially localize to tight junctions (ZO-1) while also exhibiting puncta that likely represent endosomal uptake of the particles. In contrast, only puncta are visible in the cells exposed to the 24.7 nm nanoparticles (FIG. 10D-10F). Experiments were repeated on a separate set of Caco-2 cells with a different passage and similar results were observed (FIGS. 11A-11F). The higher permeability of ultrasmall C′ dots across the Caco-2 membranes can likely be attributed to both passive (i.e. through cell-cell junctions) and active (i.e. through the cells) transport mechanisms, while the 24.7 nm nanoparticles only participate in an active transport mechanism. To better understand the mechanism by which these particles traverse the Caco-2 cell monolayer, the Caco-2 permeability experiments were repeated using the 6.3 nm and 24.7 nm particles. The cells were fixed, the ZO-1 tight junction protein was immunostained with Alexa Fluor 488-antibody conjugates, and fluorescence laser scanning confocal microscopy was performed to visualize the localization of these particles in relation to the tight junctions. From FIGS. 10A-10C, the ultrasmall C′ dots (Cy5) preferentially localize to tight junctions (Alexa Fluor 488) while also exhibiting puncta that likely represent endosomal uptake of the particles. In contrast, only puncta are visible in the cells exposed to the 24.7 nm nanoparticles (FIGS. 10D-10F). Experiments were repeated on a separate set of Caco-2 cells with a different passage and similar results were observed (FIGS. 11A-11F). The higher permeability of ultrasmall C′ dots across the Caco-2 membranes can likely be attributed to both passive (i.e. through cell-cell junctions) and active (i.e. through the cells) transport mechanisms, while the 24.7 nm nanoparticles only participate in an active transport mechanism. In all, these results suggests that PEGylated silica nanoparticles with sizes<20 nm, particularly with ultrasmall sizes below 10 nm, could be viable candidates for oral administration.

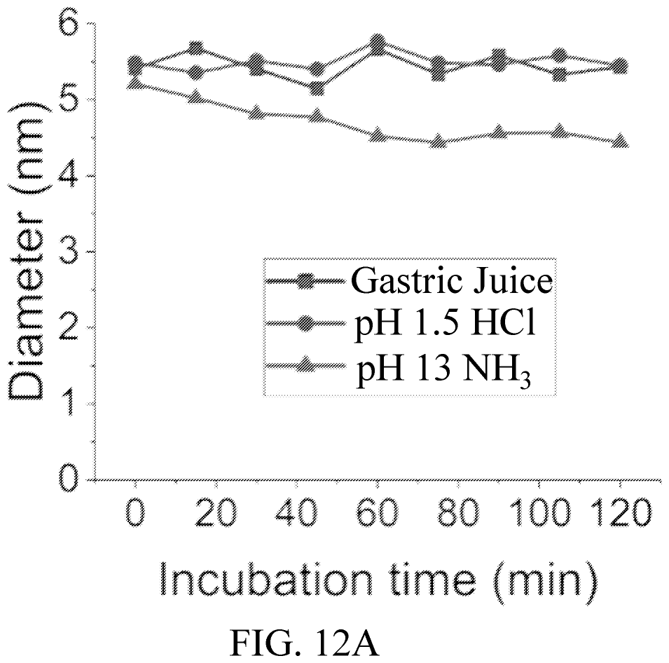

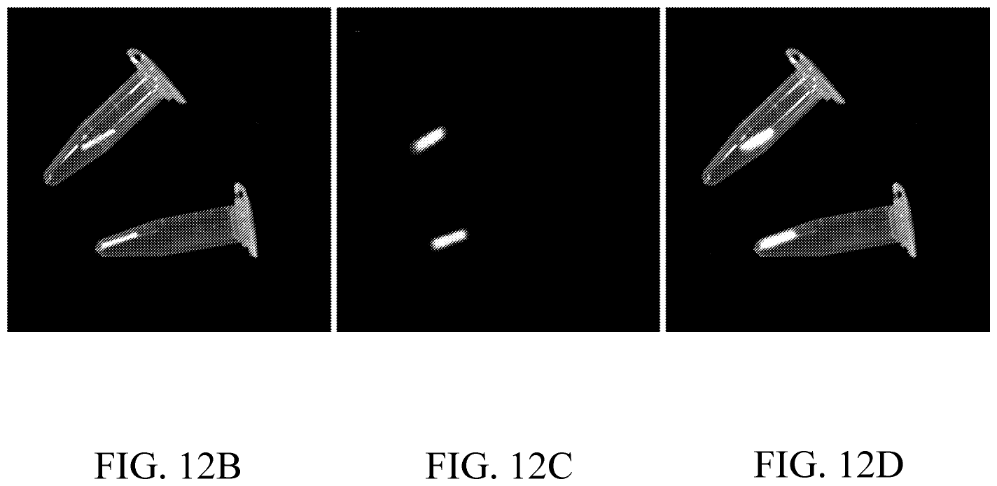

Nanoparticle Stability in Gastric Juice. Prior to reaching the small intestine, orally administered nanoparticles would have to traverse the acidic environment of the stomach. To study the ability of ultrasmall C′ dots to survive conditions found in the stomach, we studied their stability in artificial gastric juice, pH 1.5 HCl, and pH 13 NH₃. Artificial gastric juice (pH˜1.5) is an aqueous solution mainly comprised of HCl, NaCl, and pepsin and is made to simulate the contents of the stomach. For comparison, pH 1.5 HCl was used to demonstrate purely acidic pH effects on particle stability and pH 13 NH₃ was employed as a positive control as basic pH conditions are known to dissolve silica. 100 μL of 120 μM Cy5-C′ dots were diluted into 300 μL of either gastric juice, HCl, or NH₃ solutions and placed on a tube rotator at 37° C. Small aliquots were taken every 15 minutes over a 2-hour period, on which confocal FCS was applied to track the particle size over time. These parameters were chosen by taking into account the maximum volume of gastric acid in a mouse stomach, the total internal volume of a mouse stomach, and the expected residence time within the stomach. From FIG. 12A, ultrasmall C′ dots exhibit no significant size change over the 2-hour period in both the gastric juice and pH 1.5 HCl, while their diameter decreased by ˜0.8 nm within that time when exposed to pH 13 NH₃. Silica has previously been shown to be metastable at pH<4, but to dissolve at pH>11, so these findings were not unexpected. These data suggests that C′ dots in their standard formulation, without any further protection, are already able to survive the stomach's acidic environment and therefore should be suitable for oral administration.

Nanoparticle Pill Formulation. As a final step towards the translation of ultrasmall C′ dots to a formulation suitable for oral administration, a procedure to encapsulate C′ dots in a pill was generated (FIGS. 12B-12D). This is particularly important for scenarios when C′ dots are carrying sensitive payloads that may be degraded by the acidic environment of the stomach. In short, maltodextrin was used as a matrix agent and added to the receiving chamber of a gelatin pill. C′ dots suspended in 70% ethanol were then added, and the mixture allowed to form a water-soluble gel. The pill was then closed, and food-grade shellac used to coat the final, nanoparticle-loaded pill. The food grade shellac acts as an enteric coating that protects the nanoparticle formulation from the low pH environment of the stomach. It was shown that this nanoparticle pill formulation does in fact encapsulate C′ dots and does not inhibit their fluorescent properties by obtaining fluorescence images in an IVIS imaging system equipped with a 660 nm emission filter (FIGS. 12C-12D). It was further shown that this nanoparticle pill formulation exhibits negligible particle release at a pH similar to that of the stomach but significantly releases C′ dots at a pH similar to what is found in the small intestine. This was accomplished by placing pills into PBS with a pH adjusted to ˜1.5 for 3 hours and then to PBS with pH˜7.4 for 1 hour. Every 30 minutes, a small aliquot of PBS was collected and the Cy5 absorbance observed with a UV/Vis spectrometer. In all, this suggests that the pill formulation and coating could be suitable for protecting C′ dots and act as capable vessels for oral particle delivery.

In this work it was demonstrated that ultrasmall fluorescent and PEGylated core-shell silica nanoparticles exhibit properties that appear conducive to oral administration. A pill formulation was further developed that could protect sensitive payloads attached to the C′ dot surface from the acidic stomach environment. By developing a simple method of mucus reconstitution from porcine gastric mucin and employing an imaging FCS technique using a quasi (or leaky)-TIRF microscopy set-up, it was first experimentally determined the diffusion coefficients, D_{mucus}, of silica nanoparticles of sizes ranging from ˜5-50 nm suspended in highly scattering mucus. When plotting the ratio of D_{mucus}/D_{water}, which signifies how fast a nanoparticle diffuses in mucus relative to water, two distinct regimes of particle behavior were observed with two different apparent slopes and a cutoff nanoparticle diameter of ˜20 nm beyond which particle diffusion slowed down substantially. Furthermore, using the classic Caco-2 permeability assay, it was observed that smaller nanoparticles were able to permeate the cell monolayer more readily than larger ones. Similar to what was found in the diffusion studies, silica nanoparticles<20 nm in diameter, and in particular with sizes below the cutoff for renal clearance below 10 nm, showed the most promise as they had significant and detectible permeation likely attributed to both passive and active transport mechanisms. The observation that PEGylated silica nanoparticles, particularly ultrasmall C′ dots, were capable of permeating the Caco-2 monolayer with permeabilities above 1×10⁻⁶ cm/s is significant as this has been one of the most significant factors limiting the oral administration of silica nanoparticles to date. The finding that C′ dots exhibit high stability in gastric juice suggests that their standard formulation may already be suitable for oral administration without application of further protection strategies. A pill formulation was further demonstrated that would enable the oral administration of C′ dots delivering pH-sensitive payloads to the stomach, which can protect the payloads from the acidic stomach environment. Taken together, these results evidence that ultrasmall fluorescent and PEGylated core-shell silica nanoparticles (C′ dots), which are already being used in multiple human clinical trials and have potential therapeutic applications, in particular in oncology, show much promise as candidates for oral administration to patients.

Methods. Materials: Tetramethylorthosilicate (TMOS), tetraethylorthosilicate (TEOS), aminopropyltriethoxysilane (APTES), 2.0 M ammonia, l-arginine, HCl, NaOH, ethanol, methanol, DMSO, porcine gastric mucin, Lucifer Yellow, maltodextrin, and ATTO647N-maleimide were purchased from Millipore Sigma; 2-[methoxy(polyethyleneoxy)6-9propyl]trimethoxysilane (mPEG(6-9)-silane) and 3-mercaptopropyltrimethoxysilane (MPTMS) were purchased from Gelest; Cy5-maleimide and Cy5-NHS ester were purchased from Lumiprobe; RPMI 1640, Hank's balanced salt solution (HBSS), and phosphate buffered saline (PBS) were purchased from Life Technologies; 30 kDa MWCO spin filters and Sephacryl 400 HR resin were purchased from GE Healthcare; 96 well Transwell inserts and fetal bovine serum (FBS) were purchased from Corning; goat serum was purchased from Gibco; Rabbit anti-ZO1 primary antibody and Alexa Fluor 488 goat anti-rabbit IgG secondary antibody were purchased from Abcam; artificial gastric juice was purchased from Carolina Biological Supply Company.

Particle synthesis: Cy5 and ATTO647N C′ dots were synthesized as previously described. Nanoparticles with final diameters of 6.3 nm and 10.0 nm were synthesized with Cy5 and those with diameters of 18.9 nm and 24.7 nm were synthesized with ATTO647N. Briefly, a mono functional maleimido derivatized dye was dissolved in DMSO overnight in a glovebox. A 25-fold excess of mercaptopropyltrimethoxysilane (MPTMS) was added to the dissolved dye and allowed to react overnight in the glove box. The next day a flask containing deionized water adjusted to pH 8 using 2.0 M ammonia in ethanol solution was prepared and stirred vigorously. Tetramethylorthosilicate (TMOS) and the prepared dye-silane conjugate were added to the flask and allowed to react overnight. For particles with additional shells, for each shell, 500 μL of TEOS diluted in DMSO (⅕v/v) was added in 100 doses over 25 hours until the desired size was reached. The following day, 100 μL of mPEG(6-9)-silane was added to the flask and allowed to react overnight. The following day the stirring of the solution was stopped, and the flask was heated to 80° C. for 24 hours. Following this the particles were extensively dialyzed using 10 k MWCO cellulose dialysis tubing, followed by syringe filtration with a 200 nm membrane, spin filtering with a 30 k MWCO PES membrane spin filter and finally GPC purification through Superdex 200 resin on a Bio-Rad FPLC. The particles were then characterized using fluorescence correlation spectroscopy (FCS) on a home-built setup and UV/Vis spectroscopy on a Cary 5000 spectrometer. ATTO647N-encapsulating particles had a slight adjustment to this synthesis protocol: For a 10 mL reaction 2 mL of 0.02 M NH4OH solution and 8 mL of DI water were added to the reaction flask instead of the previously reported 1 mL. The resultant increase in pH was used to decrease the final size of the nanoparticles, as dyes with positive charges tend to form larger nanoparticles under standard conditions.

Large Particle Synthesis: To form the silica cores, 135 μL of TEOS was added to the top surface of a solution of 8.16 mL of DI water and 1.16 mL of 10 mg/mL l-arginine that was heated to 60° C. and stirred at 150 rpm. Approximately 8 hours after the addition another 135 μL of TEOS was added to the top surface and allowed to react overnight. The following day another 135 μL of TEOS was added to the top surface and 8 hours later another 135 μL of TEOS was added to the top surface of the solution. On the third day of the reaction, 143.5 μL of TEOS was added to the top surface of the reaction and separately Cy5 dye was conjugated with a silane. To that end, Cy5-NHS ester at a concentration of 12.6 μM was mixed thoroughly with 5.2 μL of a solution of 10 μL of APTES dissolved in 90 μL of DMSO. The conjugation was allowed to take place overnight. On day 4 of the synthesis 2 mL of the core solution and all of the Cy5 conjugation mixture were added to 8 mL of DI water, then 143.5 μL of TEOS was added to the top surface of the solution and allowed to react overnight. The following day another 143.5 μL of TEOS was added to the top surface of the solution and allowed to react overnight. This process could be repeated to grow larger nanoparticles. When the reaction was to be terminated, the day after the last addition of TEOS a 2 mL aliquot of the solution was taken and put in 8 mL of DI water. The pH was adjusted to 9-10 using ammonium hydroxide and the solution stirred at 600 rpm. After the solution was stirring, 100 μL of mPEG(6-9)-silane was added to the solution and allowed to stir for an additional 6 hours. Finally, the stirring was stopped, and the solution was heated to 80° ° C. overnight.

Gel Permeation Chromatography (GPC): Preparative scale gel permeation chromatography was carried out on a Bio-Rad FPLC equipped with a UV detector set to 275 nm. Particles were purified in isocratic mode using 0.9 wt. % NaCl in deionized water. The solution was prepared at the time of purification by diluting 0.2 μm membrane filtered 5 M NaCl in water (Santa Cruz Biotechnology) with deionized water. The column used was hand-packed with either Superdex 200 resin for smaller particles or Sephacryl 400 resin for large particles with dimensions 20 mm×300 mm and run at a flow rate of 2.0 mL/min. All samples were concentrated in GE Life Sciences 30 kDa MWCO VivaSpin filters prior to injection, and the total injection volume was less than 1 mL per run. Particles eluted around the 15-minute mark and the total run lasted 30 minutes. Fractions within the full width at half maximum (FWHM) of the peak˜15 min were collected to separate out the particles from aggregates and unreacted precursors.

Confocal FCS: All confocal FCS measurements were carried out on a homebuilt confocal FCS setup. In short, a continuous wave laser beam (633 nm solid state laser) was focused onto the image plane of a water immersion microscope objective (Zeiss Plan-Neofluar 63× NA 1.2). The stokes-shifted emitted fluorescence was collected by the same objective, passed through a dichroic mirror, spatially filtered by a 50 μm pinhole, spectrally filtered by long pass filters (ET6651lp, Chroma), and detected by an avalanche photodiode detector (SPCM-AQR-14, PerkinElmer). Respective autocorrelation curves were fitted accounting for translational diffusion and photo-induced cis-trans isomerization in the case of Cy5 and singlet-triplet transition in the case of ATTO647N by using equation (2).

$$\\ \begin{matrix}G ( τ ) = 1 + \frac{1}{{N}_{m}} ( \frac{1}{1 + τ / {τ}_{D}} ) {( \frac{1}{1 + τ / ( {τ}_{D} {κ}^{{}_{} 2} )} )}^{1 / 2} \frac{1}{( 1 - P )} ( 1 - P + P exp ( τ / {τ}_{P} ) ) & ( 2 )\end{matrix}$$

where Nₘ is the number of dye molecules or particles in the ellipsoidal observation volume, defined by a structure factor κ=ω_{z}/ω_{xy} with axial (ω_{z}) and radial (ω_{xy}) radii. τ_{D} is the average diffusion time of a dye or particle through the observation volume. P is the fraction of Cy5 dye molecules in the non-fluorescent cis-conformation or ATTO647N dyes in the triplet state, which exhibit the characteristic relaxation time τₚ. All correlation curves were normalized according to equation (3).

G(τ)=(G(τ)−1)Nₘ (3)

The FCS observation volume, V_{eff}, was calibrated before each measurement by diluting a dye stock solution with DI water to nanomolar concentrations and determining the structure factor with a standard dye (Alexa Fluor 647). All measurements were carried out at 5 kW cm⁻². All FCS samples were measured five times in five individual 30 s runs in a 35 mm glass bottom dish (P35G-1.5-10-C, Mattek Corporation) at nanomolar concentration in DI water at 20° C. The particle diameters, d, were determined from the fits using equations (4) and (5).

$$\\ \begin{matrix}D = \frac{{ω}_{xy}^{{}_{} 2}}{4 {τ}_{D}} & ( 4 )\end{matrix}$$

$$\\ \begin{matrix}d = 2 \frac{{k}_{B} T}{6 π η D} & ( 5 )\end{matrix}$$

where D is the diffusion constant, k_{B} is the Boltzmann's constant, and T is the absolute temperature. Since cis-trans isomerization of co-diffusion Cy5 molecules is independent from each other, the amplitude of isomerization, α, appears smaller, for particles carrying more than one Cy5 molecule. Therefore, P needs to be adjusted for the average number of dyes per particle, nₘ, using equations (6) and (7).

P=α/(1+α) (6)

α=αₚnₘ (7)

To determine nₘ the measured optical density of each sample was compared to the mean particle concentration as obtained by FCS using equation (8).

$$\\ \begin{matrix}{n}_{m} = \frac{{C}_{Abs}}{{〈 C {}_{} 〉}_{FCS}} & ( 8 )\end{matrix}$$

Imaging FCS: The instrumentation of Imaging FCS was described previously. The quasi-TIRF mode was manually created by introducing the excitation beam at an angle slightly smaller than the critical angle. A stack of 20,000 images from a 50×50 pixel (pixel size in the image plane=160 nm) region of interest (ROI) were recorded by an EMCCD camera (Andor iXon 897, Andor Technology) at a frame rate of 3.5 ms/frame. The images were first 10×10 binned to create hyper-pixels (hyper-pixel size=1.6 μm) and fluorescence fluctuations from these binned hyper-pixels were used to create raw autocorrelation functions (ACFs) which were then fitted with equation (9):

$$\\ \begin{matrix}G ( τ ) = \frac{1}{N} {( \frac{erf ( p ( τ ) ) + \frac{( {e}^{- {( p ( τ ) )}^{2} - 1} )}{\sqrt{π} p ( τ )}}{erf ( \frac{a}{{ω}_{0}} ) + \frac{{ω}_{0}}{a \sqrt{π}} ( {e}^{- \frac{{a}^{2}}{{ω}_{0}^{2}}} - 1 )} )}^{2} + {G}_{∞} ; & ( 9 )\end{matrix}$$

$$\\ p ( τ ) = \frac{a}{\sqrt{4 D τ + {ω}_{0}^{{}_{} 2}}}$$

where G(τ) is the ACF as a function of lag time (τ), N is the number of particles diffusing within the hyper-pixel, D is the diffusion coefficient in the hyper-pixel, a is the length of hyper-pixel in the object plane (1.6 μm), ω₀ is the point spread function (PSF) of the microscope, and G∞ is the convergence value of G(τ) at very large lag times. N, D and G∞ were used as fit parameters, and do was experimentally determined using the method described previously. All analyses were done using a FIJI/ImageJ plug-in for Imaging FCS (Imaging_FCS 1.491).

Imaging Well PEGylation: The glass surface of a 35 mm glass bottom dish was plasma etched and then PEG(6-9)-silane diluted in pH˜8 aqueous solution was added to the surface. The dish was then incubated in a water bath at 70° C. overnight. The dish was finally washed with PBS three times.

Mucus Preparation: Porcine gastric mucin was hydrated in PBS (pH 7.5) for 72 hours at 4° C. The final solution was 5% by weight porcine gastric mucin. The mucin and PBS were shaken vigorously for 72 hours to ensure all the mucin was rehydrated. After 72 hours, the pH of the rehydrated mucus was taken and adjusted to ˜7.4 using 2.5 M NaOH to better approximate the mucus found in the intestine. The mucus was used within 24 hours of preparation and was stored at 4° C. when not in use.

Rheometry: An Anton Paar MCR 301 rheometer was employed for all rheometric analysis. Complex viscosity, elastic modulus, and viscous modulus were determined by dynamic oscillatory viscosity measurements performed on reconstituted mucus samples under constant strain of 20%. The protocol employed a 25 mm diameter cone and plate geometry with 0.1-degree cone angle. The sample was sinusoidally deformed over the frequency range 10⁻¹10⁻² rads/s. Measurements were performed at 25° C.

Caco-2 Cell Culture: Caco-2 cells (ATCC) were cultured in RPMI 1640 supplemented with 20% fetal bovine serum and 10k/10k penicillin/streptomycin (Lonza). 75 μL of Caco-2 cells were plated on the apical side of a 96-well Transwell dish at a seeding density of 2×10⁵ cells/mL. 75 μL of media was used on the apical side and 235 μL on the basolateral side. The media was changed every other day for a week, followed by changes every day until the Pₐₚₚ of a Lucifer Yellow assay was <5×10⁻⁷ cm/s. Cells were incubated at 37° C. with 5% CO₂ and 90% humidity.

Lucifer Yellow Monolayer Integrity Assay: Lucifer Yellow (LY) was dissolved at a concentration of 0.1 mg/mL in Hank's balanced salt solution (HBSS). 75 μL of LY solution was added to the apical side and 235 μL HBSS was added to the basolateral side. The plate was incubated for 1 hour and then the basolateral was collected. The concentration of LY on the basolateral side was determined using fluorescence 488/520 ex/em with a Spectramax M2. This concentration was used to calculate the Pₐₚₚ with equation 1 described herein. Cell Groups 1, 2, and 3 were used for the 6.3 nm, 24.7 nm, and 33.9 nm nanoparticles, respectively.

Nanoparticle Caco-2 Permeability Experiment: Nanoparticles with diameters of 6.3 nm, 24.7 nm, and 33.9 nm were suspended in HBSS at a concentration of 5 μM as determined by confocal FCS. 75 μL were added to the apical side and 235 μL HBSS was added to the basolateral side, with the basolateral side being collected after 1 hour incubation. The nanoparticle concentration on the basolateral side was determined using confocal FCS as described above and equation 1 was used to calculate the Pₐₚₚ. 3 wells of Caco-2 monolayers were used for each nanoparticle size. Cell Groups 1, 2, and 3 were used for the 6.3 nm, 24.7 nm, and 33.9 nm nanoparticles, respectively.

Fluorescence Microscopy: Caco-2 monolayers were exposed to 6.3 nm or 24.7 nm nanoparticles as described above. The cells were then washed 3 times with PBS and fixed with ice cold methanol for 5 minutes. Next, they were exposed to blocking solution (5% goat serum in PBS) for 1 h at room temperature followed by labeling with rabbit anti-ZO1 primary antibody (1:200) for 1 h at room temperature. Cells were subsequently washed three times with PBS followed by exposure to Alexa Fluor 488 goat anti-rabbit IgG secondary antibodies (1:1000) in blocking solution at room temperature for 1 h. After washing three times with PBS, the Transwell inserts were removed and mounted onto a glass slide with a #1.5 coverslip with PBS. Images were collected on a Zeiss LSM 710 Confocal Microscope using a 63× objective. The Cy5 encapsulated by the nanoparticles and the Alexa Fluor 488 labeling ZO-1 were excited using the 633 nm and 488 nm laser lines, respectively.

Pill Formation: 6.3 nm nanoparticles were solvent exchanged into 70% ethanol using a 30 kDa MWCO spin filter to a final concentration of 45 uM. Then maltodextrin powder was packed into the receiving chamber of a porcine gelatin pill casing (Braintree Scientific) followed by the addition of the nanoparticle solution until a matrix was formed. The cap of the pill casing was then placed over the receiving chamber. Food-grade shellac (PME) was finally sprayed around the exterior of the loaded pill to seal it closed.

Pill Fluorescence Imaging: Pills inside 1.5 mL microcentrifuge tubes were imaged in an IVIS Spectrum imaging system. They were excited using a DsRed excitation filter and the emission was collected through a 660 nm filter with a 0.5 s exposure time.

Pill Dissolution Experiments: Phosphate buffered saline (PBS, Quality Biologic) was pH adjusted to 1.5 using HCl. Pills were placed in 2 mL of this solution for 3 hours and 100 μL was collected every hour. These pills were then placed into 2 mL of standard PBS with pH 7.4 for 3 hours with 100 uL collections every hour. The concentration of particles in the collected solutions were observed by taking the UV/Vis spectrum of each sample. A total of 3 pills were studied in this experiment.

Although the present disclosure has been described with respect to one or more particular embodiments and/or examples, it will be understood that other embodiments and/or examples of the present disclosure may be made without departing from the scope of the present disclosure.

1. A method of oral delivery, the method comprising:
orally administering one or more composition(s) to an individual, each composition comprising a plurality of silica nanoparticles and/or aluminosilicate nanoparticles, wherein each silica nanoparticle or aluminosilicate nanoparticle independently comprises a silica core or an aluminosilicate core, respectively, and a plurality of polyethylene glycol (PEG) groups disposed on at least a portion of a surface or surfaces of the silica nanoparticle or the aluminosilicate nanoparticle, respectively, wherein the silica nanoparticles and/or the aluminosilicate nanoparticles have a longest linear dimension of about 20 nm or less, and wherein at least a portion of the silica nanoparticles and/or the aluminosilicate nanoparticles are delivered to the post-stomach portion of the gastrointestinal tract of the individual.

2. The method of claim 1, wherein the silica nanoparticles and/or the aluminosilicate nanoparticles have a longest linear dimension of about 7 nm or less.

3. The method of claim 1, wherein at least a portion of the silica nanoparticles and/or the aluminosilicate nanoparticles pass through the mucus layer and permeate the epithelial lining of the post-stomach portion of the gastrointestinal tract of the individual.

4. The method of claim 1, wherein the silica nanoparticles and/or the aluminosilicate nanoparticles exhibit one or more or all of the following:
a diffusion coefficient through the mucus layer (D_{mucus}) of from about 5 μm²/s to about 35 μm2/s;
a diffusion coefficient through water (D_{water}) of from about 20 μm²/s to about 70 μm²/s; or
a ratio of the diffusion coefficients through the mucus layer and water (D_{mucus}/D_{water}) of from about 0.1 to about 0.5.

5. The method of claim 1, wherein at least a portion of the silica nanoparticles and/or the aluminosilicate nanoparticles comprise one or more targeting group(s), one or more diagnostic group(s), one or more therapeutic group(s), or any combination thereof, disposed on at least a portion of a surface or surfaces of the silica nanoparticles and/or the aluminosilicate nanoparticles and/or within at least a portion of the silica matrix or the aluminosilicate matrix of individual silica nanoparticles or aluminosilicate nanoparticles, respectively.

6. The method of claim 5, wherein at least a portion of the silica nanoparticles and/or the aluminosilicate nanoparticles delivered to the post-stomach portion of the gastrointestinal tract of the individual retain at least a portion of one or more targeting activit(ies), one or more diagnostic activit(ies), one or more therapeutic activit(ies), or any combination thereof, of the silica nanoparticles and/or the aluminosilicate nanoparticles.

7. The method of claim 1, wherein the composition(s) further comprise(s) one or more material(s) that render the composition(s) suitable for delivery of at least a portion of the silica nanoparticles and/or the aluminosilicate nanoparticles to the post-stomach portion of the gastrointestinal tract of the individual.

8. The method of claim 7, wherein the plurality of silica nanoparticles and/or aluminosilicate nanoparticles is disposed within the material(s).

9. The method of claim 7, wherein at least a portion of the material(s) comprise(s) one or more enteric material(s).

10. The method of claim 1, wherein the individual is an individual in need of targeting, diagnosing, treating, preventing, or any combination thereof, a current or potential disease, disease state, condition, disorder, side effect, or any combination thereof, and wherein the current or potential disease, disease state, condition, disorder, side effect, or any combination thereof, is targeted, diagnosed, treated, prevented, or any combination thereof, in the individual.

11. A method of targeting, diagnosing, treating, preventing, or any combination thereof, a current or potential disease, disease state, condition, disorder, side effect, or any combination thereof, in an individual, the method comprising:
orally administering one or more composition(s) to the individual, the composition(s) comprising a plurality of silica nanoparticles and/or aluminosilicate nanoparticles, wherein each silica nanoparticle or aluminosilicate nanoparticle independently comprises a silica core or an aluminosilicate core, respectively, and a plurality of PEG groups disposed on at least a portion of a surface or surfaces of the silica nanoparticle or the aluminosilicate nanoparticle, respectively, wherein the silica nanoparticles and/or the aluminosilicate nanoparticles have a longest linear dimension of about 20 nm or less, wherein at least a portion of the silica nanoparticles and/or the aluminosilicate nanoparticles are delivered to the post-stomach portion of the gastrointestinal tract of the individual and the current or potential disease, disease state, condition, disorder, side effect, or any combination thereof, is targeted, diagnosed, treated, prevented, or any combination thereof.

12. The method of claim 11, wherein the silica nanoparticles and/or the aluminosilicate nanoparticles have a longest linear dimension of about 7 nm or less.

13. The method of claim 11, wherein the current or potential disease, disease state, condition, disorder, side effect, or any combination thereof, is chosen from infections, cancers, neurological conditions/diseases, neurodegenerative diseases, psychological conditions/diseases, inflammatory conditions/diseases, cardio-vascular diseases, and any combination thereof.

14. The method of claim 11, wherein at least a portion of the silica nanoparticles and/or the aluminosilicate nanoparticles comprise one or more targeting group(s), one or more diagnostic group(s), one or more therapeutic group(s), or any combination thereof, disposed on at least a portion of a surface or surfaces of the silica nanoparticles and/or the aluminosilicate nanoparticles and/or within at least a portion of the silica matrix or the aluminosilicate matrix of individual silica nanoparticles or aluminosilicate nanoparticles, respectively.

15. The method of claim 11, wherein at least a portion of the silica nanoparticles and/or the aluminosilicate nanoparticles delivered to the post-stomach portion of the gastrointestinal tract of the individual retain at least a portion of one or more targeting activit(ies), one or more diagnostic activit(ies), one or more therapeutic activit(ies), or any combination thereof, of the silica nanoparticles and/or the aluminosilicate nanoparticles.

16. The method of claim 11, wherein the individual is an individual in need of targeting, diagnosing, treating, preventing, or any combination thereof, of the current or potential disease, disease state, condition, disorder, side effect, or any combination thereof, and wherein the current or potential disease, disease state, condition, disorder, side effect, or any combination thereof, is targeted, diagnosed, treated, prevented, or any combination thereof, in the individual.

17. The method of claim 14, wherein the diagnostic group(s) is/are independently at each occurrence chosen from dye group(s), sensor group(s), radioisotope(s), and any combination thereof, and wherein the method further comprises obtaining one or more image(s) of the individual.

18. The method of claim 11, the method further comprising one or more additional targeting modalit(ies), one or more additional therapeutic modalit(ies), one or more additional diagnostic modalit(ies), or any combination thereof.

19. The method of claim 11, wherein the composition(s) further comprise(s) one or more material(s) that render the composition(s) suitable for delivery of the silica nanoparticles and/or the aluminosilicate nanoparticles to the post-stomach portion of the gastrointestinal tract of the individual.

20. The method of claim 19, wherein the plurality of silica nanoparticles and/or aluminosilicate nanoparticles is disposed within the material(s).

21. The method of claim 19, wherein at least a portion of the material(s) comprise(s) one or more enteric material(s).

22. An oral delivery composition comprising:
a plurality of silica nanoparticles and/or aluminosilicate nanoparticles, wherein each silica nanoparticle or aluminosilicate nanoparticle independently comprises a silica core or an aluminosilicate core, respectively, and a plurality of polyethylene glycol (PEG groups disposed on at least a portion of a surface or surfaces of the silica nanoparticle or the aluminosilicate nanoparticle, respectively, wherein the silica nanoparticles and/or the aluminosilicate nanoparticles have a longest linear dimension of about 20 nm or less; and
one or more material(s) that render(s) the composition suitable for delivery of at least a portion of the silica nanoparticles and/or the aluminosilicate nanoparticles to the post-stomach portion of the gastrointestinal tract of an individual to whom the composition has been orally administered.

23. The oral delivery composition of claim 22, wherein the silica nanoparticles and/or the aluminosilicate nanoparticles have a longest linear dimension of about 7 nm or less.

24. The oral delivery composition of claim 22, wherein the aluminosilicate nanoparticles comprise an Al/Si atomic ratio of from about 0.01% to about 30%.

25. The oral delivery composition of claim 22, wherein at least a portion of the silica nanoparticles and/or the aluminosilicate nanoparticles comprise one or more targeting group(s), one or more diagnostic group(s), one or more therapeutic group(s), or any combination thereof, disposed on at least a portion of or all of a surface or surfaces of the silica nanoparticles and/or the aluminosilicate nanoparticles and/or within at least a portion of the silica matrix or the aluminosilicate matrix of individual silica nanoparticles or aluminosilicate nanoparticles, respectively.

26. The oral delivery composition of claim 25, wherein one or more of the PEG groups, one or more of the targeting group(s), one or more of the diagnosing group(s), one or more of the therapeutic group(s), or any combination thereof, is/are covalently bound via a linking group to one or more of the silica nanoparticles and/or the aluminosilicate nanoparticles.

27. The oral delivery composition of claim 25, wherein one or more of the PEG groups comprise(s) one or more of the targeting group(s), one or more of the diagnosing group(s), one or more of the therapeutic group(s), or any combination thereof.

28. The oral delivery composition of claim 25, wherein the targeting group(s) is/are independently at each occurrence chosen from antibody fragment(s), aptamer(s), protein(s)/peptide(s) c), nucleic acid(s), and any combination thereof.

29. The oral delivery composition of claim 25, wherein the diagnostic group(s) is/are independently at each occurrence chosen from dye group(s), sensor group(s), radioisotope(s), and any combination thereof.

30. The oral delivery composition of claim 25, wherein the therapeutic group(s) is/are independently at each occurrence chosen from drug(s), nucleic acid(s), biological material(s), radioisotope(s), and any combination thereof.

31. The oral delivery composition of claim 22, wherein the composition further comprises one or more material(s) that render the composition(s) suitable for delivery of at least a portion of the silica nanoparticles and/or the aluminosilicate nanoparticles to the post-stomach portion of the gastrointestinal tract of the individual to whom the composition has been orally administered.

32. The oral delivery composition of claim 31, wherein at least a portion of the material(s) comprise(s) one or more enteric material(s).

33. The oral delivery composition of claim 31, wherein at least a portion of the material(s) comprise(s) one or more polymeric material(s).

34. The oral delivery composition of claim 33, wherein the at least a portion of the polymeric material(s) is/are one or more pH-sensitive polymeric material(s).

35. The oral delivery composition of claim 31, wherein the material(s) coat the plurality of silica nanoparticles and/or aluminosilicate nanoparticles.

36. The oral delivery composition of claim 22, wherein the composition further comprises one or more additional material(s) that render the composition suitable for sustained silica nanoparticle and/or aluminosilicate nanoparticle release, delayed silica nanoparticle and/or aluminosilicate nanoparticle release, controlled silica nanoparticle and/or aluminosilicate nanoparticle release, time-dependent silica nanoparticle and/or aluminosilicate nanoparticle delivery, or any combination thereof.

37. The oral delivery composition of claim 22, wherein the composition further comprises one or more one or more pharmaceutically acceptable excipient(s).

38. The oral delivery composition of claim 22, wherein the composition is in the form of a pill, a capsule, a tablet, a dragée, a bead, or a granule.
